(19) United States
(12) Patent Application Publication
(10) Pub. No.: US 2025/0268811 A1
(43) Pub. Date: Aug. 28, 2025

(54) DISPERSION COMPRISING A POLYMERIC PARTICLE, A STABILIZER BEARING A C9-C22 ALKYL GROUP, AN OIL AND WATER, AND PROCESS FOR TREATING KERATIN MATERIALS USING THE DISPERSION

(71) Applicant: L'OREAL, Paris (FR)

(72) Inventors: Philippe ILEKTI, CHEVILLY LARUE (FR); Simon TAUPIN, AULNAY-SOUS-BOIS (FR); Julien PORTAL, AULNAY-SOUS-BOIS (FR)

(21) Appl. No.: 18/245,411

(22) PCT Filed: Dec. 15, 2021
(86) PCT No.: PCT/EP2021/085928

§ 371 Date: Mar. 15, 2023

(30) Foreign Application Priority Data
Dec. 18, 2020 (FR) FR2013701

(51) Int. Cl. A61K 8/81; A61K 8/04; A61K 8/31; A61Q 1/06; A61Q 1/10; A61Q 5/06

(52) U.S. Cl. CPC A61K 8/8152; A61K 8/04; A61K 8/31; A61Q 1/06; A61Q 1/10; A61Q 5/06; A61K 2800/654; A61K 2800/88

(57) ABSTRACT

The present invention relates to a dispersion (A) comprising i) at least one particle consisting of an ethylenic polymer, ii) at least one polymeric stabilizer comprising a (C₉-C₂₂)alkyl group, and iii) at least one hydrocarbon-based fatty substance which is liquid at 20° C. and 1 atmosphere. The invention also relates to a process for treating keratin materials, notably human keratin materials such as the skin or the hair, involving the application to said materials of at least one dispersion (A); to a process for preparing the dispersion, and to a multi-compartment kit comprising ingredients i) to iii).

The dispersion (A) and the process for treating keratin materials as defined above make it possible to obtain a treatment for said materials which is notably resistant to shampoo washing, to sebum, to sweat and/or to water, but also to fatty substances, notably dietary fatty substances such as oils.

The present invention relates to a dispersion (A) comprising i) at least one particle consisting of an ethylenic polymer, ii) at least one polymeric stabilizer comprising a (C₉-C₂₂)alkyl group, iii) at least one hydrocarbon-based fatty substance which is liquid at 20° C. and 1 atmosphere, and iv) water. The invention also relates to a process for treating keratin materials, notably human keratin materials such as the skin, the hair, the eyelashes or the eyebrows, involving the application to said materials of at least one dispersion (A); to a process for preparing the dispersion, and to a multi-compartment kit comprising ingredients i) to iv).

During the ageing process, various signs appear on the skin which are very characteristic of this ageing, reflected notably by a change in the skin structure and functions. The main clinical signs of skin ageing are notably the appearance of fine lines and deep wrinkles, which increase with age.

It is known practice to treat these signs of ageing by using anti-ageing cosmetic compositions. However, these active agents have the drawback of being effective for the treatment of wrinkles only after repeated application. Now, it is increasingly sought to obtain an immediate effect of the active agents used, rapidly resulting in smoothing-out of wrinkles and fine lines and/or in the disappearance of fatigue marks.

Cosmetic products often require the use of a film-forming polymer to obtain a deposit of the product on keratin materials that has good cosmetic properties. In particular, it is necessary for the film-forming deposit to have good persistence, in particular for the deposit not to transfer during contact with the fingers or clothing, and also good resistance on contact with water, notably rain or during showering and for the deposit to be insensitive to perspiration or sebum, and also food fats, notably dietary fats such as oils.

It is known practice to use dispersions of polymer particles, in organic media such as hydrocarbon-based oils. Polymers are notably used as film-forming agents in makeup products such as mascaras, eyeliners, eyeshadows or lipsticks. EP 0 749 747 describes in the examples dispersions in hydrocarbon-based oils (liquid paraffin, isododecane) of acrylic polymers stabilized with polystyrene/copoly(ethylene-propylene) diblock copolymers. The film obtained after application of the dispersion to the skin is sparingly glossy. FR 1 362 795 also describes the use of dispersions of surface-stabilized polymer particles containing hydrocarbon-based oils for making up the lips and eyelashes. WO 2010/046229 describes dispersions in isododecane of acrylic polymers stabilized with stabilizing polymers. FR 1 362 795 describes the use of dispersions of surface-stabilized polymer particles containing hydrocarbon-based oils for making up the lips and the eyelashes.

In the field of dyeing keratin fibres, it is already known practice to dye keratin fibres via various techniques using direct dyes for non-permanent dyeing, or dye precursors for permanent dyeing.

Non-permanent dyeing or direct dyeing consists in dyeing keratin fibres with dye compositions containing direct dyes. These dyes are coloured and colouring molecules that have affinity for keratin fibres. They are applied to the keratin fibres for a time necessary to obtain the desired colouring, and are then rinsed out.

The standard dyes that are used are, in particular, dyes of the nitrobenzene, anthraquinone, nitropyridine, azo, xanthene, acridine, azine or triarylmethane type, or natural dyes.

Some of these dyes may be used under lightening conditions, which enables the production of colourings that are visible on dark hair.

It is also known practice to dye keratin fibres permanently via oxidation dyeing. This dyeing technique consists in applying to the keratin fibres a composition containing dye precursors such as oxidation bases and couplers. Under the action of an oxidizing agent, these precursors form one or more coloured substances in the hair.

The variety of molecules used as oxidation bases and couplers allows a wide range of colours to be obtained, and the colourings resulting therefrom are generally permanent, strong and resistant to external agents, notably to light, bad weather, washing, perspiration and rubbing.

In order to be visible on dark hair, these two dyeing techniques require prior or simultaneous bleaching of the keratin fibres. This bleaching step, performed with an oxidizing agent such as hydrogen peroxide or persalts, results in appreciable degradation of the keratin fibres, which impairs their cosmetic properties. The hair then has a tendency to become coarse, more difficult to disentangle and more brittle.

Another dyeing method consists in surface dyeing, notably using pigments. Specifically, the use of pigment on the surface of keratin fibres generally makes it possible to obtain colourings that are notably visible on dark hair, since the surface pigment masks the natural colour of the fibre. The use of pigment for dyeing keratin fibres is described, for example, in patent application FR 2 741 530, which recommends using, for the temporary dyeing of keratin fibres, a composition comprising at least one dispersion of film-forming polymer particles including at least one acid function and at least one pigment dispersed in the continuous phase of said dispersion.

The colourings obtained via this dyeing method are generally sparingly persistent and are quite rapidly lost on shampoo washing.

It is moreover known practice from patent application FR 2 907 678 to perform coloured coating of the hair using a composition comprising a polysiloxane/polyurea block copolymer and a pigment. However, with such a composition, the coating results obtained may occasionally lack homogeneity and the strand separation after combing is not always satisfactory.

FR 3 014 875 describes the use of a dispersion of C₁-C₄ alkyl (meth)acrylate polymer particles surface-stabilized with an isobornyl (meth)acrylate polymeric stabilizer in a non-aqueous medium containing an oil. The deposits obtained using this technology are not always satisfactory, notably in terms of resistance to sebum.

FR 3 029 786 is focused on makeup dispersions of polymer particles stabilized with at least one stabilizer which is a C₈ alkyl (meth)acrylate homopolymer or a copolymer of C₈ alkyl (2-ethylhexyl) (meth)acrylate and of C₁-C₄ alkyl (meth)acrylate. These dispersions are not always satisfactory in terms of resistance to the fatty substances of sebum, which may be a curb on their use in lip makeup, for example. Furthermore, these dispersions may have a feel that is considered too “tacky” after application to the keratin materials and that may be prohibitive for certain applications such as lip or eyelash makeup.

Thus, the aim of the present invention is to provide a composition for treating keratin materials, in particular the skin, preferably human skin and more preferentially facial skin, which is not tacky, which has good persistence with respect to external attacking factors, and over time, does not leach, and is resistant to sweat and sebum and insensitive to oils such as dietary oils. Furthermore, the composition may comprise cosmetic active agents such as those for obtaining a skin-tensioning effect, for caring for the body, the face and the hair, for protecting against ultraviolet (UV), or for making up the face, the lips, the eyelashes, the eyebrows and the hair. Said composition may notably be intended for care and/or makeup, notably for making up the lips.

Moreover, consumers are in search of water-based presentation forms, containing as few volatile compounds as possible. For example, in the hair field, it is always desirable, whenever possible, to replace volatile compounds with water in the compositions used, notably in hair salons. Environmental challenges are also tending towards minimizing the use of volatile compounds. Thus, one of the aims of the present invention is to provide a composition with a reduced content of volatile compounds. Another aim is to provide a film-forming composition containing an aqueous phase, affording access to a wider formulation range. It is also sought to use as few surfactants as possible, or even none at all, in the cosmetic composition for environmental reasons, but also for the purpose of avoiding potential long-term damage to keratin materials.

Another aim of the present invention is to provide a composition for treating keratin fibres, notably human keratin fibres such as the hair, the eyelashes or the eyebrows, which has good resistance to attacking factors such as brushing, does not leach, is resistant to sweat, sebum, light and bad weather, and is persistent with respect to shampoo washing and to the various attacking factors to which said fibres may be subjected, without degrading said fibres, and while keeping the keratin fibre strands perfectly separated.

The technical problem has been solved by the use of a dispersion (A) for treating keratin materials, in particular a) keratin fibres, notably human keratin fibres such as the hair, the eyelashes and the eyebrows or B) human skin, in particular of the lips, in which the dispersion (A) comprises:
i) one or more particles including one or more polymers chosen from:
   a) ethylenic homopolymers of (C₁-C₄)alkyl (C₁-C₄)(alkyl)acrylate, preferably (C₁-C₄)alkyl (meth)acrylate;
   b) ethylenic copolymers of b1) (C₁-C₄)alkyl (C₁-C₄)(alkyl)acrylate, and of b2) ethylenic monomers comprising one or more groups from among carboxyl, anhydride, phosphoric acid, sulfonic acid and/or aryl such as benzyl; in particular, b2) is a (C₁-C₄)(alkyl) acrylic acid, more particularly copolymers of (C₁-C₄)alkyl (meth)acrylate and of (meth)acrylic acid;
   c) ethylenic copolymers of (C₁-C₄)alkyl (C₁-C₄)(alkyl)acrylate, preferably (C₁-C₄)alkyl (meth)acrylate; and
ii) one or more polymeric stabilizers chosen from:
   d) ethylenic homopolymers of (C₈-C₂₂)alkyl (C₁-C₆)(alkyl)acrylate, preferably ethylenic homopolymers of (C₉-C₂₂)alkyl (meth)acrylate; and
   e) ethylenic copolymers of e1) (C₉-C₂₂)alkyl (C₁-C₆)(alkyl)acrylate, and of e2) (C₁-C₄)alkyl (C₁-C₄)(alkyl)acrylate, preferably copolymers of (C₉-C₂₂)alkyl (meth)acrylate and of (C₁-C₄)alkyl (meth)acrylate;
iii) one or more hydrocarbon-based liquid fatty substances;
iv) water; and
v) optionally one or more cosmetic active agents chosen from f) dyes, g) pigments; h) active agents for caring for keratin materials, notably the skin, and j) UV (A) and/or (B) screening agents, and also m) mixtures thereof;
it being understood that the amount of water in the dispersion (A) is greater than or equal to 2% by weight and less than 50% by weight relative to the total weight of the dispersion, particularly the amount of water in the dispersion is between 5% and 49% by weight relative to the total weight of the dispersion, more particularly between 10% and 47% by weight, between 15% and 48% by weight, preferentially between 18% and 45%, even more preferentially between 20% and 40% by weight, relative to the total weight of the dispersion (A).

More particularly, the subject of the invention relates to the use of the dispersion (A) as defined previously for treating keratin materials, notably human keratin materials such as the hair, the eyelashes, the eyebrows or the skin, preferably for dyeing keratin fibres and/or for shaping keratin fibres such as the hair, or for making up the skin or for skincare or for providing a non-coloured immediate effect.

A subject of the invention is also the dispersion (A) as defined previously, and also a process for treating keratin materials, preferably a) keratin fibres, notably human keratin fibres such as the hair, the eyelashes or the eyebrows, or B) human skin, in particular of the lips, comprising the application to said materials of a dispersion (A), as defined previously. A subject of the invention is also a kit or device comprising several compartments comprising the ingredients i) to v) as defined previously.

A subject of the invention is also a composition, preferably a cosmetic composition, comprising the dispersion (A) as defined previously, and the process for applying said composition to keratin materials.

A subject of the invention is also a process for treating keratin materials, preferably a) keratin fibres, notably human keratin fibres such as the hair, the eyelashes, the eyebrows or B) human skin, in particular the lips, comprising the application to said materials of at least one dispersion (A), as defined previously; preferentially, after application of the dispersion (A) to the keratin materials, the composition is left to dry on said keratin materials, either naturally, or with the aid of heating devices used in cosmetics, such as a hairdryer.

The dispersion (A) and the process for treating keratin materials as defined above make it possible to obtain a treatment for said materials which is notably resistant to shampoo washing, to sebum, to sweat and/or to water, but also to fatty substances, notably dietary fatty substances such as oils. Furthermore, the dispersion is easy to use in compositions, notably cosmetic compositions, is easy to manufacture and remains stable over time. Specifically, the dispersion (A) in accordance with the present invention makes it possible to obtain deposits that are very resistant to external attacking factors, notably to sebum and to the fatty substances found in food, in particular liquid fatty substances such as plant oils and in particular olive oil. It appears that the makeup produced with at least one dispersion (A), notably lip makeup, is particularly resistant to external attacking factors such as liquid fatty substances, in particular with respect to plant oils such as olive oil. Furthermore, the makeup results obtained with the dispersions (A) are very aesthetic and glossy. Furthermore, these dispersions of polymer particles are found at a high solids content in the hydrocarbon-based liquid fatty substance(s) iii). It appears that the application of the dispersions (A) of the invention to keratin fibres makes it possible to obtain coatings that are persistent with respect to external attacking factors (sunlight, water, shampoo washing, perspiration, sebum, etc.).

In addition, when the composition or the dispersion comprises one or more dyes and/or pigments, the coloured keratin materials have a colouring that is visible on all types of materials, notably on dark keratin materials, which is persistent with respect to soaps, shower gels or shampoos while at the same time preserving the physical qualities of the keratin material. Such a coating is in particular resistant to the external attacking factors to which the keratin fibres, notably the hair, may be subjected, such as blow-drying and perspiration. The use of dispersion (A) on keratin materials, in particular on keratin fibres, makes it possible to obtain a smooth and uniform deposit. Moreover, it has been observed, surprisingly, that the keratin fibre strands remained perfectly separated, and could be styled without any problem.

For the purposes of the present invention and unless otherwise indicated:
an “alkyl radical” is a linear or branched saturated C₁-C₈, in particular C₁-C₆, preferably C₁-C₄ hydrocarbon-based group such as methyl, ethyl, n-propyl, isopropyl, n-butyl, isobutyl and t-butyl;
a “(C₉-C₂₂)alkyl” radical is a linear or branched, preferably linear, saturated C₉-C₂₂, in particular C₁₀-C₂₀, preferentially C₁₂-C₁₈, more preferentially C₁₂-C₁₆ hydrocarbon-based group, such as lauryl (C12), myristyl (C14), hexadecyl (C16), stearyl (C18), arachidyl (C20), behenyl (C22); more particularly, (C₉-C₁₈)alkyl is a linear or branched, preferably linear, saturated C₉-C₁₈ hydrocarbon-based group;
an “alkylene radical” is a linear or branched divalent saturated C₁-C₈, in particular C₁-C₆, preferably C₁-C₄ hydrocarbon-based group such as methylene, ethylene or propylene;
a “cycloalkyl” radical is a cyclic saturated hydrocarbon-based group comprising from 1 to 3 rings, preferably 2 rings, and comprising from 3 to 13 carbon atoms, preferably between 5 and 10 carbon atoms, such as cyclopentyl, cyclohexyl, cycloheptyl, norbornyl or isobornyl, the cycloalkyl radical being able to be substituted with one or more (C₁-C₄)alkyl groups such as methyl; preferably, the cycloalkyl radical is an isobornyl group;
a “cyclic” radical is a cyclic saturated or unsaturated, aromatic or non-aromatic hydrocarbon-based group comprising from 1 to 3 rings, preferably 1 ring, and comprising from 3 to 10 carbon atoms, such as cyclohexyl or phenyl;
an “aryl” radical is a monocyclic or fused or non-fused bicyclic, unsaturated cyclic aromatic radical comprising from 6 to 12 carbon atoms; preferably, the aryl group comprises 1 ring and contains 6 carbon atoms, such as phenyl;
an “aryloxy” radical is an aryl-oxy, i.e. aryl-O—, radical, with aryl as defined previously, preferably phenoxy;
an “aryl(C₁-C₄)alkoxy” radical is an aryl-(C₁-C₄)alkyl-O-radical, preferably benzoxy;
the term “keratin materials” particularly means human skin (keratinized epithelium) and human keratin fibres such as head hair, the eyelashes, the eyebrows, and bodily hair, preferentially head hair, the eyebrows and the eyelashes, even more preferentially head hair;
the term “keratin fibres with strand separation” means keratin fibres, notably the hair, which, after application of the composition and drying, are not stuck together (or of which the strands are all separated from each other) and thus do not form clumps of fibres;
the term “insoluble monomer” thus means any monomer whose homopolymer or copolymer is not in soluble form, i.e. completely dissolved to a concentration of greater than 5% by weight at room temperature (20° C.) in said medium. However, the “insoluble” monomers may, as monomers, be soluble or insoluble in the hydrocarbon-based liquid fatty substance(s) iii), it being understood that they become insoluble after polymerization in the hydrocarbon-based liquid fatty substance(s) iii);
the term “ethylenic homopolymer” means a polymer derived from the polymerization of identical monomers;
the term “ethylenic copolymer” means a polymer derived from the polymerization of different monomers, in particular at least two different monomers. Preferably, the ethylenic copolymer of the invention is derived from two or three different monomers, more preferentially derived from two different monomers;
the term “ethylenic monomer” means an organic compound including one or more conjugated or non-conjugated unsaturations of >C═C< type, which is capable of polymerizing;
the term “soluble monomer” means any monomer whose homopolymer or copolymer, preferably homopolymer, is soluble to at least 5% by weight, at 20° C., in the hydrocarbon-based liquid fatty substance(s) iii) of the dispersion. The homopolymer is completely dissolved in the carbon-based liquid(s) iii), visually at 20° C., i.e. there is no visible sign of any deposit, or precipitate, or agglomerate, or insoluble sediment;
the term “fatty substance” means an organic compound that is immiscible in water at ordinary room temperature (25° C.) and at atmospheric pressure (760 mmHg) (solubility of less than 5%, preferably 1% and even more preferentially 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance ethanol, ether, liquid petroleum jelly or decamethylcyclopentasiloxane. These fatty substances are neither polyoxyethylenated nor polyglycerolated. They are different from fatty acids, since salified fatty acids constitute soaps that are generally soluble in aqueous media;
the term “liquid fatty substance” notably refers to a fatty substance that is liquid at 25° C. and 1 atmosphere; preferably, said fatty substance has a viscosity of less than or equal to 7000 centipoises at 20° C.;
the term “hydrocarbon-based fatty substance” means a fatty substance which comprises at least 50% by weight, notably from 50% to 100% by weight, for example from 60% to 99% by weight, or even from 65% to 95% by weight, or even from 70% to 90% by weight, relative to the total weight of said fatty substance, of carbon-based compound, having a global solubility parameter in the Hansen solubility space of less than or equal to 20 (MPa)^{1/2}, or a mixture of such compounds;
the global solubility parameter δ according to the Hansen solubility space is defined in the article “Solubility parameter values” by Grulke in the book “Polymer Handbook”, 3rd Edition, Chapter VII, pages 519-559, by the relationship δ=(d_{D}²+d_{P}²+d_{H}²)^{1/2} in which:—d_{D} characterizes the London dispersion forces arising from the formation of dipoles induced during molecular impacts,—d_{P} characterizes the Debye interaction forces between permanent dipoles,—d_{H} H characterizes the forces of specific interactions (such as hydrogen bonding, acid/base, donor/acceptor, etc.); The definition of solvents in the Hansen three-dimensional solubility space is described in the article by Hansen: The three-dimensional solubility parameters, J. Paint Technol. 39, 105 (1967);
the term “oil” means a fatty substance that is liquid at room temperature (25° C.) and at atmospheric pressure;
the term “hydrocarbon-based oil” means an oil formed essentially from, or even constituted of, carbon and hydrogen atoms, and optionally of oxygen and nitrogen atoms, and not containing any silicon or fluorine atoms. It may contain hydroxyl, ester, ether, carboxylic acid, amine and/or amide groups;
the term “volatile oil” means an oil (or non-aqueous medium) that can evaporate on contact with keratin materials, in particular the skin, in less than one hour, at room temperature and at atmospheric pressure. The volatile oil is a volatile cosmetic oil, which is liquid at room temperature, notably having a non-zero vapour pressure, at room temperature and at atmospheric pressure, in particular having a vapour pressure ranging from 0.13 Pa to 40 000 Pa (10-3 to 300 mmHg), preferably ranging from 1.3 Pa to 13000 Pa (0.01 to 100 mmHg) and preferentially ranging from 1.3 Pa to 1300 Pa (0.01 to 10 mmHg);
the term “non-volatile oil” means an oil with a vapour pressure of less than 0.13 Pa at room temperature and at atmospheric pressure;
the term “silicone oil” means an oil comprising at least one silicon atom and notably at least one Si—O group. The silicone oil may be volatile or non-volatile;
the term “dispersant” refers to a compound which can protect the dispersed particles from agglomerating or flocculating. This dispersant may be a surfactant, an oligomer, a polymer or a mixture of several thereof, bearing one or more functionalities with strong affinity for the surface of the particles to be dispersed; in particular, they can attach physically or chemically to the surface of the pigments. These dispersants also contain at least one functional group that is compatible with or soluble in the continuous medium. Said agent may be charged: it may be anionic, cationic, zwitterionic or neutral;
the term “pigment” refers to any pigment that gives colour to keratin materials, of synthetic or natural origin, the solubility of the pigments in water at 25° C. and at atmospheric pressure (760 mmHg) being less than 0.05% by weight and preferably less than 0.01%;
the term “lake” refers to dyes adsorbed onto insoluble particles, the assembly thus obtained remaining insoluble during use. The inorganic substrates onto which the dyes are adsorbed are, for example, alumina, silica, calcium sodium borosilicate or calcium aluminium borosilicate and aluminium. Among the organic dyes, mention may be made of cochineal carmine;
the term “hair dyes” refers to the oxidation dyes and direct dyes used for dyeing keratin fibres, notably human keratin fibres such as the hair;
the term “anhydrous dispersion or composition” means a dispersion or composition containing less than 2% by weight of water, or even less than 0.5% of water, and notably free of water; where appropriate, such small amounts of water may notably be provided by ingredients of the composition which may contain residual amounts thereof;
the term “pigments with special effects” refers to pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade; and
the term “submicron” or “submicronic” refers to pigments having a particle size that has been micronized by a micronization method and having a mean particle size of less than a micrometer (μm), in particular between 0.1 and 0.9 μm, and preferably between 0.2 and 0.6 μm.

### The Dispersion (A)

The dispersion (A) of the invention comprises i) one or more particles of at least one polymer that is surface-stabilized with ii) at least one stabilizer in a medium that is preferably anhydrous, also containing iii) at least one hydrocarbon-based liquid fatty substance and iv) water.

In order to obtain the dispersion (A), it is proposed to polymerize particular monomers that are capable of forming the polymeric core i) in the presence of a polymeric statistical stabilizer ii) comprising in major amount a portion ii) that is soluble and in minor amount a portion i) that is insoluble in the dispersion medium, i.e. in the mixture of hydrocarbon-based liquid fatty substance(s) iii) and iv) water.

The dispersions according to the invention consist of particles, which are generally spherical, and of at least one surface-stabilized polymer, in a mixture of hydrocarbon-based liquid fatty substance(s) iii) and iv) water.

Preferably, said particles i) are not or are sparingly crosslinked.

The polymer particles i) and the stabilizer(s) ii) are preferably in the mixture of hydrocarbon-based liquid fatty substance(s) iii) and iv) water in an amount of between 2% and 40% by weight, notably between 4% and 25% by weight of soluble monomer (the monomer forming the stabilizer(s) ii)) and between 60% and 98% by weight, notably from 75% to 96% by weight, of insoluble monomer (the monomer forming the particles i)).

In the present description, the term “soluble monomer” refers to any monomer of which the polymer, notably the homopolymer, is soluble, to 5% by weight, at 20° C. and at atmospheric pressure in the liquid hydrocarbon-based medium consisting of liquid hydrocarbon-based fatty substances iii) of the dispersion. The polymer, notably the homopolymer, is completely dissolved in the liquid carbon-based medium, visually at 20° C. (no apparent visible deposit, or any insoluble agglomerate or sediment visible to the eye).

The term “insoluble monomer” refers to any monomer of which the polymer, notably the homopolymer, is not in soluble form, i.e. not fully dissolved at a concentration of greater than 5% by weight at room temperature (20° C.) in said liquid hydrocarbon-based medium consisting of iii) liquid hydrocarbon-based fatty substances. However, the “insoluble” monomers may, as monomers, be soluble in the liquid hydrocarbon-based medium consisting of hydrocarbon-based liquid fatty substance(s) iii) of the dispersion, it being understood that they become insoluble after polymerization.

Preferably, the monomers i) that are capable of forming the polymeric core of the particle are chosen from monomers that are insoluble in the liquid hydrocarbon-based medium consisting of iii) liquid hydrocarbon-based fatty substances. The insoluble monomers notably represent 100% by weight relative to the total weight of the monomers forming the polymeric core of the particle.

### Polymer Particles i)

The particle(s) i) of dispersion (A) of the invention preferably consist of one or more polymers chosen from:
a) ethylenic homopolymers of (C₁-C₄)alkyl (C₁-C₄)(alkyl)acrylate, preferably (C₁-C₄)alkyl (meth)acrylate ethylenic homopolymers;
b) ethylenic copolymers of (C₁-C₄)alkyl (C₁-C₄)(alkyl)acrylate, preferably (C₁-C₄)alkyl (meth)acrylate, and of (C₁-C₄)(alkyl)acrylic acid, preferably (meth)acrylic acid ethylenic copolymers;
c) ethylenic copolymers of (C₁-C₄)alkyl (C₁-C₄)(alkyl)acrylate, preferably (C₁-C₄)alkyl (meth)acrylate ethylenic copolymers.

Preferably, the particle(s) i) consist of an ethylenic polymeric core derived from homopolymers a) or copolymers b) or c) as defined previously.

According to a preferred embodiment of the invention, the polymer constituting the particles i) is an ethylenic acrylate homopolymer a) resulting from the polymerization of an identical monomer of formula (I):

[Chem 1]

H₂C═C(R)—C(O)—O—R′ (I)

in which formula (I):
R represents a hydrogen atom or a (C₁-C₄)alkyl group such as methyl, and
R′ represents a (C₁-C₄)alkyl group such as methyl or ethyl, preferably a C₁-C₄ alkyl acrylate such as methyl acrylate.

According to a particular embodiment of the invention, the polymer of the particles i) is a polymer derived from C₁-C₄ alkyl (meth)acrylate monomers. The monomers are preferably chosen from methyl (meth)acrylate, ethyl (meth)acrylate, n-propyl (meth)acrylate, isopropyl (meth)acrylate, n-butyl (meth)acrylate, isobutyl (meth)acrylate and tert-butyl (meth)acrylate, and more preferentially chosen from methyl (meth)acrylate and ethyl (meth)acrylate.

Advantageously, a C₁-C₄ alkyl acrylate monomer is used. Preferentially, the monomers are chosen from methyl acrylate and ethyl acrylate.

A C₁-C₄ alkyl methacrylate monomer is also particularly used. Preferentially, the monomers are chosen from methyl methacrylate and ethyl methacrylate, more particularly methyl methacrylate.

According to a particular embodiment of the invention, the dispersion (A) includes from 2% to 40% by weight, in particular 4% to 25%, notably from 5% to 20% by weight and preferably from 6% to 10% by weight of (C₉-C₂₂)alkyl (C₁-C₆)(alkyl)acrylate monomers included in d) or e) in the hydrocarbon-based liquid fatty substance(s) iii), relative to the total weight of polymers contained in said dispersion.

According to an advantageous embodiment of the invention, the dispersion (A) includes from 60% to 98% by weight, notably from 75% to 96% of monomers a) to c) relative to the total weight of polymers contained in said dispersion.

Preferably, the monomers that are capable of forming the polymeric core of the particle i) are chosen from monomers that are insoluble in the hydrocarbon-based liquid fatty substance(s) iii) of the dispersion (A). The insoluble monomers preferably represent 100% by weight, of the total weight of the monomers forming the polymeric core of the particle.

### Ethylenic Monomers Bearing an Acid, Anhydride or Aryl Function:

According to one embodiment of the invention, the particle(s) i) include b) ethylenic copolymers of b1) (C₁-C₄)alkyl (C₁-C₄)(alkyl)acrylate and of b2) ethylenic monomers comprising one or more groups from among carboxyl, anhydride, phosphoric acid, sulfonic acid and/or aryl such as benzyl.

More particularly, the ethylenic monomer(s) comprising one or more carboxyl, anhydride, phosphoric acid, sulfonic acid and/or aryl groups are chosen from (1), (2), (3) and (4):
(1) R₁ (R₂)C═C(R₃)-Acid with R₁, R₂ and R₃ representing a hydrogen atom or a CO₂H, H₂PO₄ or SO₃H group, and Acid representing a carboxyl, phosphoric acid or sulfonic acid, preferably carboxyl; preferably, (1) represents (5) H₂C═C(R)—C(O)—O—H with R representing a hydrogen atom or a (C₁-C₄)alkyl group such as methyl;
(2) H₂C═C(R)—C(O)—N(R′)-Alk-Acid with R and R′, which may be identical or different, representing a hydrogen atom or a (C₁-C₄)alkyl group; Alk represents a (C₁-C₆)alkylene group optionally substituted with at least one group chosen from Acid as defined previously and hydroxyl; and Acid is as defined previously, preferably carboxyl or sulfonic acid;
(3) Ar—(Rₐ)C═C(R_{b})—R_{c} with Rₐ, R_{b} and R_{c}, which may be identical or different, representing a hydrogen atom or a (C₁-C₄)alkyl group, and Ar representing an aryl group, preferably benzyl, optionally substituted with at least one acid group CO₂H, H₂PO₄ or SO₃H, preferably substituted with a CO₂H or SO₃H group;
(4) maleic anhydride of formulae (4a) and (4b):

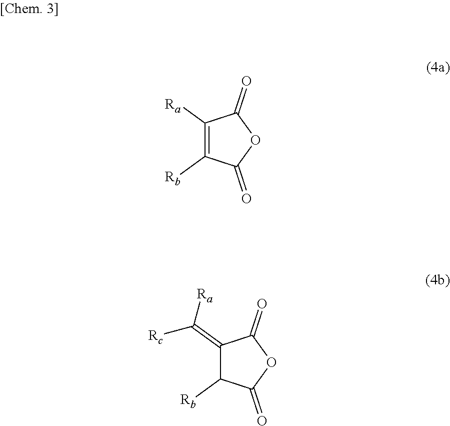

in which formulae (4a) and (4b) Rₐ, R_{b} and R_{c}, which may be identical or different, represent a hydrogen atom or a (C₁-C₄)alkyl group; preferably, Rₐ, R_{b}, and R_{c} represent a hydrogen atom. Preferentially, the ethylenically unsaturated anhydride monomer of the invention is of formula (4b) and more preferentially is maleic anhydride; and
more particularly, b1) is chosen from (1) and (4), in particular (5) and more particularly (5).

According to another particular embodiment of the invention, the polymer constituting the particles i) is an ethylenic acrylate copolymer b) derived from the polymerization:
of at least one monomer of formula (I) as defined previously, preferably a C₁-C₄ alkyl acrylate such as methyl acrylate, ethyl acrylate; and
of a monomer of formula (II)

[Chem 2]

H₂C═C(R)—C(O)—O—H (II)

in which formula (II)
R is as defined previously; in particular is acrylic acid.

According to this embodiment, the amount of acrylic acid ranges from 0.01% to 30% by weight relative to the total weight, preferably between 0.1% and 20% by weight relative to the weight of the polymer(s) of the particles i), preferably relative to the total weight of the core. More particularly, i) is in particular a copolymer derived from the copolymerization of acrylic acid with one or more C₁-C₄ alkyl (meth)acrylate monomers chosen in particular from methyl (meth)acrylate, ethyl (meth)acrylate.

According to another preferred embodiment of the invention, the polymer constituting the particles i) is an ethylenic acrylate copolymer b) derived from the polymerization:
of at least two different monomers: of formula (I) as defined previously, preferably a C₁-C₄ alkyl acrylate such as methyl acrylate, ethyl acrylate; and
optionally of a monomer of formula (II) as defined previously.

Preferably, b2) is a (C₁-C₄)(alkyl) acrylic acid; more particularly, b) is (are) copolymers of (C₁-C₄)alkyl (meth)acrylate and of (meth)acrylic acid.

More preferentially, b2) is chosen from crotonic acid, maleic anhydride, itaconic acid, fumaric acid, maleic acid, styrenesulfonic acid, vinylbenzoic acid, vinylphosphoric acid, acrylic acid, methacrylic acid, acrylamidopropanesulfonic acid, acrylamidoglycolic acid, acrylic acid and salts thereof, and even more preferentially b2) represents acrylic acid.

Preferably, only one type of polymer particle i) is used in the invention.

The polymer particle(s) i) of the dispersion (A) preferably have a number-mean size ranging from 5 to 600 nm, notably ranging from 10 to 500 nm and better still ranging from 20 to 400 nm.

The final size of the particles is preferably greater than 100 nm. In particular, they have a number-average size ranging from 100 nm to 600 nm, more particularly ranging from 150 nm to 500 nm and even more particularly ranging from 160 nm to 400 nm.

The mean size of the particles is determined via conventional methods known to those skilled in the art. A Malvern brand NanoZS model laser particle size analyser (which is particularly suitable for submicron dispersions) makes it possible to measure the size distribution of these samples. The operating principle of this type of machine is based on dynamic light scattering (DLS), also known as quasi-elastic light scattering (QELS) or photon correlation spectroscopy (PCS).

The sample is pipetted into a disposable plastic cuvette (four transparent faces, side length of 1 cm and volume of 4 mL) placed in the measuring cell. The data are analysed on the basis of a cumulant fit method which leads to a monomodal particle size distribution characterized by an intensity-weighted mean diameter d (nm) and a size polydispersity factor Q. The results may also be expressed in the form of statistical data such as D10; D50 (median), D90 and the mode.

Other particle size techniques make it possible to obtain this type of information, such as analysis of the individual tracking of particles (Nanoparticle Tracking Analysis, NTA), laser scattering (LS), acoustic extinction spectroscopy (AES) spatial-filter Doppler velocimetry or image analysis.

### The Stabilizer(s) ii)

The dispersion (A) according to the invention also comprises one or more stabilizers ii). Preferably, only one type of stabilizer ii) is used in the invention.

According to a particular embodiment of the invention, the stabilizer(s) ii) are chosen from d) ethylenic homopolymers of (C₉-C₂₂)alkyl (C₁-C₆)(alkyl)acrylate, in particular (C₉-C₂₀)alkyl (C₁-C₄)(alkyl)acrylate ethylenic homopolymers, preferably (C₉-C₂₂)alkyl (meth)acrylate ethylenic homopolymers and more preferentially (C₉-C₁₈)alkyl (meth)acrylate ethylenic homopolymers.

More particularly, the stabilizer(s) ii) consist of ethylenic polymers chosen from d) ethylenic homopolymers derived from the polymerization of monomers of formula H₂C═C(R)—C(O)—O—R″ with R representing a hydrogen atom or a (C₁-C₄)alkyl group such as methyl, and R″ representing a (C₉-C₂₂)alkyl and preferably (C₉-C₁₈)alkyl group. Preferably, R″ represents isodecyl, lauryl, stearyl or hexadecyl, more preferentially stearyl.

According to another particular embodiment of the invention, the stabilizer(s) ii) are chosen from e) ethylenic copolymers of (C₉-C₂₂)alkyl (C₁-C₆)(alkyl)acrylate and of (C₁-C₄)alkyl (C₁-C₄)(alkyl)acrylate, particularly (C₉-C₁₈)alkyl (C₁-C₄)(alkyl)acrylate and (C₁-C₄)alkyl (C₁-C₄)(alkyl)acrylate, preferably copolymers of (C₉-C₁₈)alkyl (meth)acrylate and of (C₁-C₄)alkyl (meth)acrylate.

More preferentially, the stabilizer(s) ii) are chosen from e) ethylenic copolymers of e1) (C₉-C₂₂)alkyl (C₁-C₆)(alkyl)acrylate and of e2) (C₁-C₄)alkyl (C₁-C₄)(alkyl)acrylate, preferably copolymers e1) of (C₉-C₂₂)alkyl (meth)acrylate and of e2) (C₁-C₄)alkyl (meth)acrylate, more particularly chosen from ethylenic copolymers e1) of monomers of formula (IV) and e2) of monomers of formula (III)

[Chem 4]

H₂C═C(R)—C(O)—O—R′ (III)

[Chem 5]

H₂C═C(R)—C(O)—O—R″ (IV)

in which formulae (III) and (IV):
R, which may be identical or different, represents a hydrogen atom or a (C₁-C₄)alkyl group such as methyl;
R′, which may be identical or different, represents a (C₁-C₄)alkyl group such as methyl or ethyl; and
R″ represents a (C₉-C₂₂)alkyl, preferably (C₁₀-C₂₀)alkyl and in particular (C₂ₙ)alkyl group with n being an integer equal to 5, 6, 7, 8, 9 or 10. Preferably, R″ represents isodecyl, lauryl, stearyl, hexadecyl or behenyl, more preferentially stearyl.

Preferentially, the stabilizer(s) ii) are chosen from copolymers derived from monomers chosen from isodecyl, lauryl, stearyl, hexadecyl or behenyl (meth)acrylates, more particularly stearyl (meth)acrylate (even more preferentially stearyl methacrylate), and C₁-C₄ alkyl (meth)acrylate, preferably methyl (meth)acrylate and/or ethyl (meth)acrylate.

More preferentially, the stabilizer(s) ii) are chosen from copolymers e) derived from monomers chosen from e1) isodecyl, lauryl, stearyl and hexadecyl (meth)acrylates, more particularly stearyl (meth)acrylate (even more preferentially stearyl methacrylate), and e2) C₁-C₄ alkyl (meth)acrylate, preferably ethyl (meth)acrylate, more particularly ethyl acrylate.

Preferably, the copolymer e) complies with the weight ratio e1)/e2) which is greater than 4.5. Advantageously, said weight ratio ranges from 5 to 15 and more preferentially said weight ratio ranges from 5.5 to 12.

Particularly, the stabilizer ii) is chosen from:
the isodecyl, lauryl, stearyl, hexadecyl or behenyl, preferably stearyl, (meth)acrylate homopolymer d), and
statistical copolymers of e1) isodecyl, lauryl, stearyl, hexadecyl or behenyl, preferably stearyl, (meth)acrylate and of e2) C₁-C₄ alkyl (meth)acrylate preferably present in a weight ratio e1)/e2) of greater than 4.5. Advantageously, said weight ratio ranges from 5 to 15 and more preferentially said weight ratio ranges from 5.5 to 12.

According to another embodiment, the stabilizer(s) ii) are chosen from the ethylenic copolymers e) derived from the polymerization e1) of a monomer of formula (IV) as defined previously and e2) of two different monomers of formula (III) as defined previously.

Preferentially, the stabilizer(s) ii) are chosen from copolymers derived from the polymerization of e1) one monomer of formula (IV) as defined previously notably chosen from isodecyl, lauryl, stearyl, hexadecyl or behenyl, preferably stearyl, (meth)acrylates and e2) of two different monomers of formula (III) as defined previously, notably different C₁-C₄ alkyl (meth)acrylates, preferably methyl and ethyl acrylate.

According to a particular embodiment of the invention, the weight ratio of monomer of formula (IV), notably isodecyl, lauryl, stearyl, hexadecyl or behenyl, preferably stearyl, (meth)acrylates e1)/monomer of formula (III), notably C₁-C₄ alkyl (meth)acrylate, preferably methyl and/or ethyl (meth)acrylate e2), i.e. e1)/e2), is greater than 4. Advantageously, said weight ratio e1)/e2) ranges from 5 to 15 and more preferentially said weight ratio ranges from 5.5 to 12.

According to another embodiment, the stabilizer(s) ii) are chosen from ethylenic copolymers e) derived from the polymerization e2) of a monomer of formula (III) as defined previously and e1) two different monomers of formula (IV) as defined previously.

Particularly, the stabilizer(s) ii) are chosen from e) ethylenic copolymers of e1) (C₈-C₂₂)alkyl (C₁-C₆)(alkyl)acrylate and of e2) (C₁-C₄)alkyl (C₁-C₄)(alkyl)acrylate, as defined previously in a (C₉-C₂₂)alkyl (C₁-C₆)(alkyl)acrylate/(C₁-C₄)alkyl (C₁-C₄)(alkyl)acrylate weight ratio e1)/e2) of greater than 4.

Preferentially, the stabilizer(s) ii) are chosen from copolymers derived from the polymerization of e1) two different monomers chosen from isodecyl, lauryl, stearyl, hexadecyl or behenyl, preferably stearyl, (meth)acrylates and of e2) one C₁-C₄ alkyl (meth)acrylate monomer, preferably methyl acrylate or ethyl acrylate; in particular, the isodecyl, lauryl, stearyl, hexadecyl or behenyl, preferably stearyl, (meth)acrylate/C₁-C₄ alkyl (meth)acrylate weight ratio e1)/e2) is greater than 4.5, more particularly greater than or equal to 5. Advantageously, said weight ratio e1)/e2 ranges from 4.5 to 10; more preferentially, said weight ratio ranges from 5 to 8 and more particularly from 5.5 to 7.

According to a particular embodiment of the invention, the dispersion (A) includes from 2% to 40% by weight, in particular from 3% to 30% by weight, more particularly from 4% to 25% by weight and notably from 5.5% to 20% by weight of (C₉-C₂₂)alkyl (C₁-C₆)(alkyl)acrylate monomers included in d) or e) in the hydrocarbon-based liquid fatty substance(s) iii), relative to the total weight of polymers contained in said dispersion.

The stabilizer(s) ii) as defined previously preferably comprise 80% to 100% by weight of monomer that is soluble in the liquid hydrocarbon-based fatty substances iii), notably from 85% to 95% by weight of soluble monomer, alone or as a mixture. The stabilizing (co) polymer(s) ii) particularly include between 0% and 20% by weight, notably between 5% and 15% by weight, of monomer that is insoluble in the liquid hydrocarbon-based fatty substances iii), alone or as a mixture.

Preferably, the stabilizer(s) ii) and the particle(s) i) have a number-average molecular weight (Mn) of between 1000 and 1000000 g/mol, notably between 5000 and 500000 g/mol and better still between 10000 and 300000 g/mol.

The dispersion (A) according to the invention is finally formed from polymer particles, of relatively large diameter, i.e. preferably greater than 100 nm, and leads to shiny deposits of film-forming deposits which are resistant to fatty substances at room temperature (25° C.), and which are notably advantageous for makeup and/or haircare applications.

The Hydrocarbon-Based Liquid Fatty Substance(s) iii)

The dispersion of polymer particles (A) according to the invention also comprises iii) one or more hydrocarbon-based liquid fatty substances in which said particles are dispersed.

According to a particular embodiment, the hydrocarbon-based liquid fatty substance(s) iii) of the invention are chosen from hydrocarbons, in particular alkanes, oils of animal origin, oils of plant origin, glycerides or fluorinated oils of synthetic origin, fatty alcohols, esters of fatty acids and/or of fatty alcohols, non-silicone waxes, and silicones; in particular, the liquid hydrocarbon-based fatty substance(s) are hydrocarbon-based oils, which are preferably volatile, or are a mixture of different volatile oils, preferentially chosen from isododecane and octyldodecanol, more particularly isododecane.

The hydrocarbon-based liquid fatty substances iii) are notably chosen from C₆-C₁₆ hydrocarbons or hydrocarbons comprising more than 16 carbon atoms and up to 50 carbon atoms, preferably between C₆ and C₁₆, and in particular alkanes, oils of animal origin, oils of plant origin, glycerides, fatty alcohols, fatty acid and/or fatty alcohol esters, and silicones.

It is recalled that, for the purposes of the invention, the fatty alcohols, fatty acid esters more particularly contain one or more linear or branched, saturated or unsaturated hydrocarbon-based groups comprising 6 to 50 carbon atoms, which are optionally substituted, in particular with one or more (in particular 1 to 4) hydroxyl groups. If they are unsaturated, these compounds may comprise one to three conjugated or unconjugated carbon-carbon double bonds.

As regards the C₆-C₁₆ alkanes, they are linear or branched, and possibly cyclic. Examples that may be mentioned include hexane, undecane, dodecane, tridecane or isoparaffins, such as isohexadecane, isodecane or isododecane and mixtures thereof such as the combination of undecane and tridecane, for instance Cetiol UTR. The linear or branched hydrocarbons containing more than 16 carbon atoms may be chosen from liquid paraffins, petroleum jelly, liquid petroleum jelly, polydecenes, and hydrogenated polyisobutene such as Parleam®.

Among the hydrocarbon-based liquid fatty substances iii) having an overall solubility parameter according to the Hansen solubility space of less than or equal to 20 (MPa)^{1/2}, mention may be made of oils, which may be chosen from natural or synthetic, hydrocarbon-based oils, which are optionally branched, alone or as a mixture.

According to a very advantageous embodiment, the dispersion (A) according to the invention comprises one or more liquid fatty substances which are one or more hydrocarbon-based oils. The hydrocarbon-based oil(s) may be volatile or non-volatile.

According to a preferred embodiment of the invention, the liquid hydrocarbon-based fatty substance(s) are hydrocarbon-based oils which are volatile or are a mixture of different volatile oils.

According to another particular embodiment, the hydrocarbon-based liquid fatty substance(s) iii) are a mixture of a volatile oil and a non-volatile oil such as an isododecane/octyldodecanol mixture.

The hydrocarbon-based oil may be chosen from:

hydrocarbon-based oils containing from 8 to 14 carbon atoms, and notably:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade names Isopar or Permethyl,
linear alkanes, for instance n-dodecane (C₁₂) and n-tetradecane (C₁₄) sold by Sasol under the respective references Parafol 12-97 and Parafol 14-97, and also mixtures thereof, the undecane-tridecane mixture, the mixtures of n-undecane (C₁₁) and of n-tridecane (C₁₃) obtained in examples 1 and 2 of patent application WO 2008/155 059 from the company Cognis, and mixtures thereof,
hydrocarbon-based oils of plant origin such as triglycerides constituted of fatty acid esters of glycerol, the fatty acids of which may have chain lengths ranging from C₄ to C₂₄, these chains possibly being linear or branched, and saturated or unsaturated; these oils are notably heptanoic acid or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil or musk rose oil; shea butter; or else caprylic/capric acid triglycerides, for instance those sold by the company Stearinerie Dubois or those sold under the names Miglyol 810®, 812® and 818®,
synthetic ethers containing from 10 to 40 carbon atoms,
linear or branched hydrocarbons of mineral or synthetic origin, such as petroleum jelly, polydecenes, hydrogenated polyisobutene such as Parleam®, squalane and liquid paraffins, and mixtures thereof,
esters such as the oils of formula R¹C(O)—O—R² in which R¹ represents a linear or branched fatty acid residue including from 1 to 40 carbon atoms and R² represents a hydrocarbon-based chain, which is notably branched, containing from 1 to 40 carbon atoms, on condition that R¹+R²>10, for instance purcellin oil (cetostearyl octanoate), isopropyl myristate, isopropyl palmitate, C₁₂ to C₁₅ alcohol benzoates, hexyl laurate, diisopropyl adipate, isononyl isononanoate, 2-ethylhexyl palmitate, isostearyl isostearate, 2-hexyldodecyl laurate, 2-octyldecyl palmitate, 2-octyldecyl myristate, alcohol or polyalcohol heptanoates, octanoates, decanoates or ricinoleates, for instance propylene glycol dioctanoate; hydroxylated esters such as isostearyl lactate, diisostearyl malate, 2-octyldecyl lactate; polyol esters and pentaerythritol esters,
fatty alcohols that are liquid at room temperature, bearing a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

In addition to the liquid hydrocarbon-based fatty substance, the dispersion (A) may comprise a silicone oil. If the silicone oil is in the dispersion (A), it is preferably in an amount which does not exceed 10% by weight relative to the weight of the dispersion (A), more particularly in an amount of less than 5% and more preferentially 2%.

In particular, the dispersion (A) comprises at least one liquid hydrocarbon-based fatty substance iii) chosen from:
plant oils formed by fatty acid esters of polyols, in particular triglycerides, such as sunflower oil, sesame oil, rapeseed oil, macadamia oil, soybean oil, sweet almond oil, beauty-leaf oil, palm oil, grapeseed oil, corn oil, arara oil, cottonseed oil, apricot oil, avocado oil, jojoba oil, olive oil or cereal germ oil;
linear, branched or cyclic esters containing more than 6 carbon atoms, notably 6 to 30 carbon atoms; and notably isononyl isononanoate;
   and more particularly the esters of formula R^{d}—C(O)—O—R^{e} in which R^{d} represents a higher fatty acid residue including from 7 to 19 carbon atoms and R^{e} represents a hydrocarbon-based chain including from 3 to 20 carbon atoms, such as palmitates, adipates, myristates and benzoates, notably diisopropyl adipate and isopropyl myristate;
hydrocarbons and notably volatile or non-volatile, linear, branched and/or cyclic alkanes, such as C₅-C₆₀ isoparaffins, which are optionally volatile, such as isododecane, Parleam (hydrogenated polyisobutene), isohexadecane, cyclohexane or Isopars; or else liquid paraffins, liquid petroleum jelly, or hydrogenated polyisobutylene; notably isododecane;
ethers containing 6 to 30 carbon atoms;
aliphatic fatty monoalcohols containing 6 to 30 carbon atoms, the hydrocarbon-based chain not including any substitution groups, such as oleyl alcohol, decanol, dodecanol, octadecanol, octyldodecanol and linoleyl alcohol; notably octyldodecanol;
polyols containing 6 to 30 carbon atoms, such as hexylene glycol; and
mixtures thereof.

Advantageously, the hydrocarbon-based liquid fatty substance(s) of the invention are apolar, i.e. formed solely of carbon and hydrogen atoms.

Preferably, the dispersion (A) comprises at least one apolar liquid hydrocarbon-based fatty substance iii) preferably chosen from:
linear or branched C₈-C₃₀, in particular C₁₀-C₂₀ and more particularly C₁₀-C₁₆ alkanes, which are volatile or non-volatile, preferably volatile;
non-aromatic cyclic C₅-C₁₂ alkanes, which are volatile or non-volatile, preferably volatile; and
mixtures thereof.

The liquid hydrocarbon-based fatty substance(s) are preferably chosen from hydrocarbon-based oils containing from 8 to 16 carbon atoms, in particular containing from 10 to 14 carbon atoms, which are preferably volatile, more particularly the apolar oils, described previously.

Among the branched C₈-C₁₆ and notably C₁₀-C₁₄ alkanes that are suitable for use as liquid hydrocarbon-based fatty substances iii) in the dispersion of the invention, mention may be made of:
isoalkanes of petroleum origin (also known as isoparaffins), such as isododecane (also known as 2,2,4,4,6-pentamethylheptane), isodecane and, for example, the oils sold under the Isopar or Permethyl trade names,
linear alkanes, for instance n-dodecane (C₁₂) and n-tetradecane (C₁₄) sold by Sasol under the references, respectively, Parafol 12-97 and Parafol 14-97, and also mixtures thereof, the undecane-tridecane mixture, mixtures of n-undecane (C₁₁) and of n-tridecane (C₁₃) from the company Cognis, and mixtures thereof.

Preferentially, the liquid hydrocarbon-based fatty substance(s) iii) of the invention are apolar, more particularly isododecane.

According to another advantageous embodiment of the invention, the hydrocarbon-based liquid fatty substance(s) are a mixture of non-volatile and volatile oil; preferably, the mixture comprises isododecane as volatile oil or a mixture of oils, notably of undecane and tridecane or else isononyl isononanoate or octyldodecanol.

In particular, in the mixture, the non-volatile oil is a phenyl silicone oil, preferably chosen from pentaphenyl silicone oils.

Preferably, the liquid hydrocarbon-based fatty substance(s) iii) are in the dispersion of the invention in an amount of between 15% by weight and 80% by weight, more preferentially between 20% and 60% by weight relative to the total weight of said dispersion (A).

According to a particular embodiment of the invention, the weight ratio of the sum of the ingredients [i)+ii)]/iii) is less than or equal to 1, more particularly, the mass ratio [i)+ii)]/iii) is between 0.5 and 1.

### Method for Preparing the Dispersion (A)

Without this being limiting, in general, the dispersion according to the invention may be prepared in the following manner:
The polymerization is performed in “dispersion” by precipitation of the polymer being formed, with protection of the formed particles with one or more stabilizers ii), preferably only one type of stabilizer ii) chosen from d) and e) as defined previously.
In a first step, the stabilizing polymer (or stabilizer ii)) is prepared by mixing the constituent monomer(s) of the stabilizing polymer d) or e) with a free-radical initiator, in a solvent known as the synthesis solvent, and by polymerizing these monomers; and then
In a second step, the monomers constituting the polymer of the particles i) is added to the stabilizing polymer ii) formed in the preceding step and polymerization of these added monomers is performed in the presence of the radical initiator.
In a third step, water is added and the ingredients i)+ii) are all stirred in the reactor before taking out the dispersion.

When the synthesis solvent is a non-volatile hydrocarbon-based liquid fatty substance iii), the polymerization may be performed in an apolar organic solvent (synthesis solvent), followed by adding the non-volatile hydrocarbon-based liquid fatty substance (which should be miscible with said synthesis solvent) and selectively distilling off the synthesis solvent.

The synthesis solvent may consist of hydrocarbon-based liquid fatty substance iii) combined with an additional solvent notably chosen from linear or branched hydrocarbon-based aliphatic-chain esters containing from 3 to 8 carbon atoms in total, such as ethyl acetate, methyl acetate, propyl acetate or n-butyl acetate.

At the end of step 1, when the synthesis solvent is a mixture, the additional solvent including the hydrocarbon-based aliphatic-chain esters as defined previously is removed via a method that is conventional to those skilled in the art, such as distillation. The polymers of the particles i) and the stabilizers ii) are found in the hydrocarbon-based liquid fatty substance iii).

The dispersion (A) optionally contains one or more cosmetic active agents v).

The cosmetic active agent(s) v) are chosen from f) dyes, g) pigments, h) active agents for caring for keratin materials, notably the skin, and i) UV-screening agents and also m) mixtures thereof, may be added during the first step. According to another variant, said cosmetic active agent(s) are added during the second step or after the second step.

According to another variant, said cosmetic active agent(s) are added during the third step.

A synthesis solvent which is such that the monomers of the polymeric stabilizer(s) ii) and the free-radical initiator vi) are soluble therein, and the polymer particles i) obtained are insoluble therein, so that they precipitate therein during their formation, is thus chosen.

In particular, the synthesis solvent chosen is one which is apolar, organic and volatile, preferably chosen from alkanes such as heptane, cyclohexane or isododecane, preferably isododecane.

According to another advantageous variant, use is made of a mixture of:
an apolar solvent as defined previously, notably isododecane,
a polar solvent notably chosen from esters such as (C₁-C₄)alkyl (C₁-C₄)alkylate, for example ethyl acetate.

When the synthesis solvent is a volatile hydrocarbon-based liquid fatty substance iii), the polymerization may be performed directly in said oil, which thus also acts as synthesis solvent. The monomers should also be soluble therein, as should the free-radical initiator, and the polymer of the particles i) which is obtained should be insoluble therein.

According to a particular form of the invention, the synthesis solvent is a mixture of liquid fatty substance such as iii) and notably isodecane with an additional solvent notably chosen from linear or branched hydrocarbon-based aliphatic-chain esters containing from 3 to 8 carbon atoms in total, such as ethyl acetate, methyl acetate, propyl acetate or n-butyl acetate and notably ethyl acetate, said additional solvent being chosen to have a boiling point below that of the liquid fatty substance. When the synthesis of the stabilizers ii) and of the particles i) is complete, the additional solvent is removed and a mixture of i) and ii) in the liquid fatty substance is obtained.

The monomers are preferably present in the synthesis solvent, before polymerization, in a proportion of from 15% to 45% by weight. The total amount of the monomers may be present in the solvent before the start of the reaction, or a portion of the monomers may be added gradually as the polymerization reaction proceeds.

The polymerization is preferentially performed in the presence vi) of one or more radical initiators which may be any initiator known to those skilled in the art for radical polymerization, such as peroxide or azo initiators, redox couples and photochemical initiators.

Mention may notably be made of initiators vi) such as:
peroxide, in particular chosen from tert-butyl peroxy-2-ethylhexanoate: Trigonox 21S; 2,5-dimethyl-2,5-bis(2-ethylhexanoylperoxy) hexane: Trigonox 141; tert-butyl peroxypivalate: Trigonox 25C75 from AkzoNobel; or
azo, in particular chosen from AIBN: azobisisobutyronitrile; V50: 2,2′-azobis(2-amidinopropane) dihydrochloride.

The polymerization is preferably performed at a temperature ranging from 70° C. to 110° C. and at atmospheric pressure.

The polymer particles i) are surface-stabilized, when they are formed during the polymerization, by means of the stabilizer ii) prepared beforehand.

The stabilization may be performed by any known means, and in particular by direct addition of the stabilizer ii), during the polymerization.

The stabilizer ii) is preferably also present in the mixture before polymerization of the monomers of the polymer of the particles i). However, it is also possible to add it continuously, notably when the monomers of the particles i) are also added continuously.

From 2% to 40% by weight and particularly from 3% to 30% by weight, more particularly from 4% to 25% by weight and preferably from 4.5% to 20% by weight of the stabilizer(s) may be used relative to the total weight of monomers used (stabilizers ii)+polymer particles i)).

The polymer particle dispersion (A) advantageously comprises from 30% to 65% by weight of solids relative to the total weight of said dispersion and preferably from 40% to 60% by weight relative to the total weight of said dispersion.

The composition according to the invention preferably comprises a content of solids (or active material) of polymers of particle i)+dispersing polymers ii) ranging from 10% to 80% by weight, relative to the total weight of the dispersion (A), and preferably ranging from 15% to 60% by weight, notably 20% to 50% by weight relative to the total weight of the dispersion (A).

In a particular preparation method, the statistical stabilizing polymer ii) is prepared in a first step. This stabilizing polymer is soluble in an apolar organic solvent of alkane type, such as isododecane.

Next, in a second step, the polymer particles i) are synthesized in the presence of the stabilizing polymer ii).

Preferentially, a solution of stabilizing polymer ii) in the liquid hydrocarbon-based fatty substance(s) iii) is prepared for the final dispersion, and the polymerization of the monomers which form the core of the particle is performed in the presence of this stabilizer ii).

The stabilizing polymer ii) may be prepared by radical polymerization optionally in the presence of a polymerization initiator vi) as defined previously.

In a second step, the monomers which form the core of the particle i) may be polymerized in the presence of said stabilizing polymer ii). This second step may be a conventional radical polymerization.

In a third step, water or an aqueous composition is added, preferably with stirring, at room temperature and at atmospheric pressure.

The dispersions are prepared in the presence of one or more liquid hydrocarbon-based fatty substances iii), preferably in an apolar organic solvent, in particular of alkane type such as isododecane, according to an industrially feasible process.

According to a preferred embodiment of the invention, the process is performed according to the following three steps:

For the preparation of these novel dispersions, a process was performed:

Step 1: Synthesis of the polymer particles in the hydrocarbon-based oil iii) In a first step, the polymer particles are synthesized in the hydrocarbon-based oil iii), which is preferably a volatile apolar oil.

An additional solvent may be mixed with the volatile polar hydrocarbon-based oil and will be chosen from esters with a chain containing from 3 to 8 carbon atoms in total, such as ethyl acetate, methyl acetate, propyl acetate or n-butyl acetate.

The dispersion of the polymer particles in the hydrocarbon-based oil iii) or the mixture of hydrocarbon-based oil/short-chain ester may be prepared in the following manner:

The polymerization is performed in “dispersion”, i.e. by precipitation of the polymer being formed, with protection of the particles i) formed with one or more stabilizers ii), preferably one stabilizer.

Step 1, the stabilizing polymer ii) (or stabilizer ii)) is prepared by mixing the constituent monomer(s) of the stabilizing polymer with a free-radical initiator vi), in a solvent known as the synthesis solvent, and by polymerizing these monomers;

Step 2, the constituent monomer(s) of the polymer of the particles are added to the stabilizing polymer formed in the preceding step and polymerization of these added monomers is performed in the presence of the free-radical initiator vi).

The polymerization is preferentially performed in the presence vi) of one or more free-radical initiators vi) as defined previously.

The polymerization is preferably performed at a temperature ranging from 70° C. to 110° C. and at atmospheric pressure.

The polymer particles are surface-stabilized, when they are formed during the polymerization, by means of the stabilizer.

At the end of this step 1, the short-chain ester is removed by distillation. Thus, the polymer of the particles is found in the hydrocarbon-based oil iii).

Step 3: The water is added and stirred with the mixture of polymer of the particles+volatile polar hydrocarbon-based oil before removing the dispersion from the reactor.

The dispersion (A) according to the invention finds a quite particular application in the cosmetic field, notably in the makeup field and notably in making up the eyes (mascara, eyeshadow or eyeliner) and in lipsticks.

The dispersions according to the invention are thus finally formed from polymer particles, of relatively large diameter (preferably greater than 100 nm), and give glossy film-forming deposits that are resistant to fatty substances at the observation temperature (25° C.).

Furthermore, since said dispersion is in oily medium with the presence of water, it becomes easy to formulate it in cosmetic compositions based on oily medium commonly used in cosmetics, in particular in the fatty phases of emulsions, but also in the aqueous phases of emulsions to enable the dissolution of water-soluble or hydrophilic active agents.

According to a particular embodiment of the invention, the process is performed according to the following three steps:

For the preparation of these novel dispersions, the following process may also be performed:

### Step 1: Synthesis of the Stabilizing Polymer ii)

The polymer forming the stabilizer ii) is synthesized in a volatile apolar hydrocarbon-based oil iii), and optionally at least one additional polar solvent such as esters with a chain containing from 3 to 8 carbon atoms in total, such as ethyl acetate, methyl acetate, propyl acetate or n-butyl acetate.

The polymerization is preferably performed in the presence of at least one radical initiator vi) as defined previously.

### Step 2: Addition of Water

Before the preparation of i), notably of the core of the polymer particles i), water iv) is added to the medium containing the stabilizing polymer(s) ii), the volatile apolar hydrocarbon-based oil iii) and optionally the additional solvent.

### Step 3: Synthesis of the Core of the Polymer Particles

The preparation of i) and notably of the core of the polymer particles i) is preferably performed in the presence of at least one radical initiator vi) as defined previously.

At the end of this step 3, the additional solvent(s) are removed by distillation. Thus, the polymer particles i)+ii) are found in the volatile apolar hydrocarbon-based oil iii) in the presence of water iv).

The dispersion (A) according to the invention finds an application in caring for and/or making up the skin and/or the lips and/or for the care, styling and/or dyeing of keratin fibres, preferably human keratin fibres, more preferentially the hair.

The dispersion according to the invention finds a quite particular application in the cosmetic field, notably in the makeup field and notably in lipsticks and lip glosses, and eyeshadows and mascaras.

### Water iv)

The dispersion comprises water iv) in an amount greater than or equal to 2% by weight and less than 50% by weight relative to the total weight of the dispersion; in particular, the amount of water in the dispersion is between 5% and 49% by weight relative to the total weight of the dispersion, more particularly between 10% and 47% by weight, between 15% and 48% by weight, preferentially between 18% and 45% by weight and even more preferentially between 20% and 40% by weight relative to the total weight of the dispersion.

According to a particular embodiment of the invention, the weight ratio of the hydrocarbon-based liquid fatty substance(s) iii)/the water v) is between 0.2 and 10, more particularly between 0.5 and 8, preferably between 0.6 and 7, more preferentially between 0.7 and 6.

The water that is suitable for use in the invention may be tap water, distilled water, spring water, a floral water such as cornflower water and/or a mineral water such as Vittel water, Lucas water or La Roche Posay water and/or a thermal water.

The dispersion (A) may also comprise one or more water-miscible solvents.

According to the present invention, the term “water-miscible solvent” denotes a compound that is liquid at room temperature and water-miscible (miscibility with water of greater than 50% by weight at 25° C. and atmospheric pressure).

The water-miscible solvents that may be used in dispersion (A) of the invention may also be volatile.

Among the water-miscible solvents that may be used in the composition according to the invention, mention may notably be made of lower monoalcohols containing from 2 to 5 carbon atoms such as ethanol and isopropanol, and glycols containing from 3 to 8 carbon atoms such as hexylene glycol, propylene glycol, 1,3-butylene glycol and dipropylene glycol.

The water may also comprise any water-soluble or water-dispersible compound that is compatible with an aqueous phase, such as associated film-forming polymers, surfactants, and mixtures thereof.

The term “surfactant” means a “surface agent”, which is a compound that is capable of modifying the surface tension between two surfaces; surfactants are amphiphilic molecules, i.e. they contain two parts of different polarity, one lipophilic and apolar, and the other hydrophilic and polar. The surfactants may be nonionic, anionic, amphoteric or cationic active agents.

According to a preferred embodiment of the invention, the dispersion (A) of the invention does not comprise more than 3% by weight of surfactants relative to the total weight of the dispersion, preferentially not more than 2% by weight of surfactants relative to the total weight of the dispersion, more particularly not more than 1% by weight of surfactants relative to the total weight of the dispersion; even more preferentially, the composition does not comprise more than 0.5% by weight of surfactants relative to the total weight of the dispersion, and better still the mixture does not comprise any surfactant.

### The Cosmetic Active Agent(s) v)

According to a particular embodiment of the invention, the dispersion (A) of the invention comprises one or more cosmetic active agents chosen from f) dyes, g) pigments; h) active agents for caring for keratin materials, and i) UV (A) and/or (B) screening agents, and also m) mixtures thereof.

According to a preferred embodiment of the present invention, the cosmetic active agent(s) of the invention are chosen from g) pigments.

According to a particular embodiment of the present invention, the cosmetic active agent(s) of the invention are chosen from h) active agents for caring for keratin materials, preferably skincare active agents.

According to yet another particular embodiment of the present patent application, the cosmetic active agent(s) of the invention are chosen from i) UV (A) and/or UV (B) screening agents, and a mixture thereof.

According to a particular embodiment of the invention, the dispersion (A) comprises iv) one or more cosmetic active agents chosen from pigments.

The pigment(s) more particularly represent from 0.5% to 40% by weight and preferably from 1% to 20% by weight relative to the total weight of the dispersion (A).

The pigments are white or coloured solid particles which are naturally insoluble in the hydrophilic and lipophilic liquid phases usually employed in cosmetics or which are rendered insoluble by formulation in the form of a lake, where appropriate. More particularly, the pigments have little or no solubility in aqueous-alcoholic media.

The pigments that may be used are notably chosen from the organic and/or mineral pigments known in the art, notably those described in Kirk-Othmer's Encyclopedia of Chemical Technology and in Ullmann's Encyclopedia of Industrial Chemistry. Pigments that may notably be mentioned include organic and mineral pigments such as those defined and described in Ullmann's Encyclopedia of Industrial Chemistry “Pigments, Organic”, 2005 Wiley-VCH Verlag Gmbh & Co. KGaA, Weinheim 10.1002/14356007.a20 371 and ibid, “Pigments, Inorganic, 1. General” 2009 Wiley-VCH Verlag Gmbh & Co. KGaA, Weinheim10.1002/14356007.a20_243.pub3.

These pigments may be in pigment powder or paste form. They may be coated or uncoated.

The pigments may be chosen, for example, from mineral pigments, organic pigments, lakes, pigments with special effects such as nacres or glitter flakes, and mixtures thereof.

The pigment may be a mineral pigment. The term “mineral pigment” refers to any pigment that satisfies the definition in Ullmann's encyclopaedia in the chapter on inorganic pigments. Among the mineral pigments that are useful in the present invention, mention may be made of iron oxides, chromium oxides, manganese violet, ultramarine blue, chromium hydrate, ferric blue and titanium oxide.

The pigment may be an organic pigment.

The term “organic pigment” refers to any pigment that satisfies the definition in Ullmann's encyclopaedia in the chapter on organic pigments.

The organic pigment may notably be chosen from nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane and quinophthalone compounds.

In particular, the white or coloured organic pigments may be chosen from carmine, carbon black, aniline black, azo yellow, quinacridone, phthalocyanine blue, the blue pigments codified in the Colour Index under the references CI 42090, 69800, 69825, 74100, 74160, the yellow pigments codified in the Colour Index under the references CI 11680, 11710, 19140, 20040, 21100, 21108, 47000, 47005, the green pigments codified in the Colour Index under the references CI 61565, 61570, 74260, the orange pigments codified in the Colour Index under the references CI 11725, 45370, 71105, the red pigments codified in the Colour Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names:
Cosmenyl Yellow IOG: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may be composed notably of particles including a mineral core, at least one binder for attaching the organic pigments to the core, and at least one organic pigment which at least partially covers the core.

The organic pigment may also be a lake. The term “lake” means dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium.

Among the dyes, mention may be made of carminic acid. Mention may also be made of the dyes known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of pigments with special effects exist: those with a low refractive index, such as fluorescent or photochromic pigments, and those with a higher refractive index, such as nacres, interference pigments or glitter flakes.

Examples of pigments with special effects that may be mentioned include nacreous pigments such as mica covered with titanium or with bismuth oxychloride, coloured nacreous pigments such as mica covered with titanium and with iron oxides, mica covered with iron oxide, mica covered with titanium and notably with ferric blue or with chromium oxide, mica covered with titanium and with an organic pigment as defined previously, and also nacreous pigments based on bismuth oxychloride. Nacreous pigments that may be mentioned include the nacres Cellini sold by BASF (mica-TiO₂-lake), Prestige sold by Eckart (mica-TiO₂), Prestige Bronze sold by Eckart (mica-Fe₂O₃) and Colorona sold by Merck (mica-TiO₂—Fe₂O₃).

Mention may also be made of the gold-coloured nacres sold notably by the company BASF under the name Brilliant gold 212G (Timica), Gold 222C (Cloisonne), Sparkle gold (Timica), Gold 4504 (Chromalite) and Monarch gold 233X (Cloisonne); the bronze nacres sold notably by the company Merck under the name Bronze fine (17384) (Colorona) and Bronze (17353) (Colorona) and by the company BASF under the name Super bronze (Cloisonne); the orange nacres sold notably by the company BASF under the name Orange 363C (Cloisonne) and Orange MCR 101 (Cosmica) and by the company Merck under the name Passion orange (Colorona) and Matte orange (17449) (Microna); the brown nacres sold notably by the company BASF under the name Nu-antique copper 340XB (Cloisonne) and Brown CL4509 (Chromalite); the nacres with a copper tint sold notably by the company BASF under the name Copper 340A (Timica); the nacres with a red tint sold notably by the company Merck under the name Sienna fine (17386) (Colorona); the nacres with a yellow tint sold notably by the company BASF under the name Yellow (4502) (Chromalite); the red nacres with a gold tint sold notably by the company BASF under the name Sunstone G012 (Gemtone); the pink nacres sold notably by the company BASF under the name Tan opale G005 (Gemtone); the black nacres with a gold tint sold notably by the company BASF under the name Nu antique bronze 240 AB (Timica), the blue nacres sold notably by the company Merck under the name Matte blue (17433) (Microna), the white nacres with a silvery tint sold notably by the company Merck under the name Xirona Silver, and the golden-green pink-orange nacres sold notably by the company Merck under the name Indian summer (Xirona), and mixtures thereof.

Still as examples of nacres, mention may also be made of particles including a borosilicate substrate coated with titanium oxide.

Particles comprising a glass substrate coated with titanium oxide are notably sold under the name Metashine MC1080RY by the company Toyal.

Finally, examples of nacres that may also be mentioned include polyethylene terephthalate glitter flakes, notably those sold by the company Meadowbrook Inventions under the name Silver 1P 0.004×0.004 (silver glitter flakes). It is also possible to envisage multilayer pigments based on synthetic substrates, such as alumina, silica, calcium sodium borosilicate, calcium aluminium borosilicate and aluminium.

The pigments with special effects may also be chosen from reflective particles, i.e. notably from particles whose size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. more luminous points that contrast with their environment, making them appear to sparkle.

The reflective particles may be selected so as not to significantly alter the colouring effect generated by the colouring agents with which they are combined, and more particularly so as to optimize this effect in terms of colour rendition. They may more particularly have a yellow, pink, red, bronze, orange, brown, gold and/or coppery colour or tint.

These particles may have varied forms and may notably be in platelet or globular form, in particular in spherical form.

The reflective particles, whatever their form, may or may not have a multilayer structure and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically.

When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or mineral materials.

More particularly, it may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Reflective particles are notably described in JP-A-09188830, JP-A-10158450, JP-A-10158541, JP-A-07258460 and JP-A-05017710.

Again as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the names Crystal Star GF 550 and GF 2525 by this same company.

Use may also be made of particles comprising a metal substrate, such as silver, aluminium, iron, chromium, nickel, molybdenum, gold, copper, zinc, tin, magnesium, steel, bronze or titanium, said substrate being coated with at least one layer of at least one metal oxide, such as titanium oxide, aluminium oxide, iron oxide, cerium oxide, chromium oxide, silicon oxides and mixtures thereof.

Examples that may be mentioned include aluminium powder, bronze powder or copper powder coated with SiO₂ sold under the name Visionaire by the company Eckart.

Mention may also be made of pigments with an interference effect which are not attached to a substrate, such as liquid crystals (Helicones HC from Wacker) or interference holographic glitter flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colours, and also particular optical effects such as metallic effects or interference effects.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

The dispersant serves to protect the dispersed particles against their agglomeration or flocculation. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C₈ to C₂₀ fatty acid and of polyols such as glycerol or diglycerol are used, such as poly(2-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the composition may be surface-treated with an organic agent.

Thus, the pigments surface-treated beforehand that are useful in the context of the invention are pigments which have been completely or partially subjected to a surface treatment of chemical, electronic, electrochemical, mechanochemical or mechanical nature with an organic agent, such as those described notably in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polyethylene;

(meth)acrylic polymers, for example polymethyl methacrylates; polymers and copolymers containing acrylate units; alkanolamines; silicone compounds, for example silicones, notably polydimethylsiloxanes; organofluorine compounds, for example perfluoroalkyl ethers; fluorosilicone compounds.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in patent U.S. Pat. No. 4,578,266.

An organic agent covalently bonded to the pigments will preferably be used.

The agent for the surface treatment may represent from 0.1% to 50% by weight relative to the total weight of the surface-treated pigment, preferably from 0.5% to 30% by weight and even more preferentially from 1% to 20% by weight relative to the total weight of the surface-treated pigment.

Preferably, the surface treatments of the pigments are chosen from the following treatments:
a PEG-silicone treatment, for instance the AQ surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a magnesium myristate treatment, for instance the MM surface treatment sold by LCW;
an aluminium dimyristate treatment, such as the MI surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrogenosiloxane/perfluoroalkyl phosphate treatment, for instance the FS01 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.

According to a particular embodiment of the invention, the dispersant is present with organic or mineral pigments in submicron-sized particulate form in the dye composition.

According to one embodiment, the dispersant and the pigment(s) are present in an amount (dispersant: pigment) of between 1:4 and 4:1, particularly between 1.5:3.5 and 3.5:1 or better still between 1.75:3 and 3:1.

The dispersant(s) may thus have a silicone backbone, such as silicone polyether and dispersants of amino silicone type other than the alkoxysilanes described previously. Among the suitable dispersants that may be mentioned are:
amino silicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxyl groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of amino silicone type other than the alkoxysilanes described previously and are cationic.

Preferably, the pigment(s) are chosen from mineral, mixed mineral-organic or organic pigments.

In one variant of the invention, the pigment(s) according to the invention are organic pigments, preferentially organic pigments surface-treated with an organic agent chosen from silicone compounds. In another variant of the invention, the pigment(s) according to the invention are mineral pigments.

The dispersion (A) may comprise one or more f) dyes, in particular one or more direct dyes.

The term “direct dye” means natural and/or synthetic dyes, other than oxidation dyes. These are dyes that will spread superficially on the fibre.

They may be ionic or nonionic, preferably cationic or nonionic.

Examples of suitable direct dyes that may be mentioned include azo direct dyes; (poly) methine dyes such as cyanines, hemicyanines and styryls; carbonyl dyes; azine dyes; nitro (hetero) aryl dyes; tri (hetero) arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (V) and (VI) and the azo cationic dyes (VII) and (VIII) below:

[Chem. 6] Het⁺-C(Rₐ)═N—N(R_{b})—Ar, Q⁻ (V)

[Chem. 7] Het⁺-N(Ra)—N═C(Rb)—Ar, Q- (VI)

[Chem. 8] Het⁺—N═N—Ar, Q- (VII)

[Chem. 9] Ar+—N═N—Ar″, Q- (VIII)

in which formula (V) to (VIII):
Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
Art represents an aryl radical, such as phenyl or naphthyl, bearing an exocyclic cationic charge, preferentially ammonium, particularly tri (C₁-C₈)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di) (C₁-C₈)(alkyl)amino optionally substituted on the alkyl group(s) with a hydroxyl group, iv) aryl(C₁-C₅)alkylamino, v) optionally substituted N—(C₁-C₈)alkyl-N-aryl(C₁-C₈)alkylamino or alternatively Ar represents a julolidine group;
Ar″ represents an optionally substituted (hetero) aryl group, such as phenyl or pyrazolyl, which are optionally substituted, preferentially with one or more (C₁-C₅)alkyl, hydroxyl, (di) (C₁-C₈)(alkyl)amino, (C₁-C₅)alkoxy or phenyl groups;
Ra and Rb, which may be identical or different, represent a hydrogen atom or a (C₁-C₈)alkyl group, which is optionally substituted, preferentially with a hydroxyl group;
   or else the substituent Ra with a substituent of Het⁺ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q-represents an organic or mineral anionic counterion, such as a halide or an alkyl sulfate.

In particular, mention may be made of the azo and hydrazono direct dyes bearing an endocyclic cationic charge of formulae (V) to (VIII) as defined previously, more particularly the cationic direct dyes bearing an endocyclic cationic charge described in patent applications WO 95/15144, WO 95/01772 and EP 714 954, preferentially the following direct dyes:

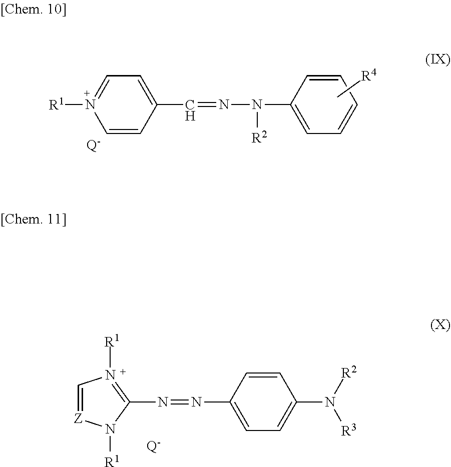

in which (IX) and (X) formulae:
R¹ represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₅)alkyl, optionally substituted (C₁-C₈)alkoxy, or (di) (C₁-C₈)(alkyl)amino optionally substituted on the alkyl group(s) with a hydroxyl group; particularly, R⁴ is a hydrogen atom,
Z represents a CH group or a nitrogen atom, preferentially CH,
Q⁻ is an anionic counterion as defined previously, in particular a halide, such as chloride, or an alkyl sulfate, such as methyl sulfate or mesyl.

In particular, the dyes of formulae (IX) and (X) are chosen from Basic Red 51, Basic Yellow 87 and Basic Orange 31 or derivatives thereof with Q-being an anionic counterion as defined previously, particularly halide such as chloride, or an alkyl sulfate such as methyl sulfate or mesyl.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

According to one embodiment of the invention, the dyes are liposoluble. They are chosen, for example, from Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice or methylene blue.

Preferably, the cosmetic active agent(s) v) are chosen from the following pigments: carbon black, iron oxides, notably black iron oxides, and micas coated with iron oxide, red iron oxides (iron (III) oxide, also known as ferric oxide), triarylmethane pigments, notably blue and violet triarylmethane pigments, such as Blue 1 Lake, azo pigments, notably red azo pigments, such as D&C Red 7, the alkali metal salts of lithol red, such as the calcium salt of lithol red B.

According to a particular embodiment of the invention, the amount of pigments ranges from 0.5% to 40% and preferably from 1% to 20% relative to the weight of the dispersion (A) comprising them.

According to a particular embodiment of the invention, the dispersion (A) comprises v) one or more cosmetic active agents chosen from oxidation dyes.
The oxidation dyes are generally chosen from one or more oxidation bases, optionally combined with one or more coupling agents.

By way of example, the oxidation bases are chosen from para-phenylenediamines, bis(phenyl)alkylenediamines, para-aminophenols, ortho-aminophenols and heterocyclic bases and the corresponding addition salts, optionally combined with coupling agents; they may particularly be chosen from meta-phenylenediamines, meta-aminophenols, meta-diphenols, naphthalene-based coupling agents and heterocyclic coupling agents and also the corresponding addition salts;
direct dyes, notably azo direct dyes; (poly) methine dyes such as cyanines, hemicyanines and styryls; carbonyl dyes; azine dyes; nitro (hetero) aryl dyes; tri (hetero) arylmethane dyes; porphyrin dyes; phthalocyanine dyes and natural direct dyes, alone or in the form of mixtures. The direct dyes may be anionic, cationic or neutral;
natural dyes, notably chosen from hennotannic acid, juglone, alizarin, purpurin, carminic acid, kermesic acid, purpurogallin, protocatechaldehyde, indigo, isatin, curcumin, spinulosin, apigenidin and orcein, and also extracts or decoctions containing these natural dyes.

The dye(s) f) more particularly represent from 0.001% to 10% by weight relative to the total weight of the dispersion (A) and preferably from 0.005% to 5% by weight relative to the total weight of the dispersion (A).

Preferably, the pigment(s) g) of the invention are chosen from carbon black, iron oxides, notably red, brown or black iron oxides, and micas coated with iron oxide, triarylmethane pigments, notably blue and violet triarylmethane pigments, such as Blue 1 Lake, azo pigments, notably red azo pigments, such as D&C Red 7, an alkali metal salt of lithol red such as the calcium salt of lithol red B; more preferentially, the pigment(s) used are chosen from red iron oxides and azo pigments, notably red pigments such as D&C Red 7.

According to a particular embodiment of the invention, the amount of pigments ranges from 0.5% to 40% and preferably from 1% to 20% relative to the weight of the dispersion (A) comprising them.

### Process for Treating Keratin Materials Using the Dispersion (A):

According to an advantageous variant of the invention, the process of the invention is a process for treating keratin fibres, in particular human keratin fibres, preferably the hair, which involves the application to said fibres of at least one dispersion (A) as defined previously.

According to a particular embodiment of the invention, after applying the dispersion (A) to the keratin materials, the composition is left to dry on said keratin materials, either naturally or using heating devices used in cosmetics, such as a hairdryer.

According to a particular embodiment of the invention, the keratin fibre treatment process is a treatment for shaping said fibres.

More particularly, the keratin fibre treatment process includes at least one shaping step, notably:
in a first step of the process, the keratin fibres are shaped using a conventional shaping means, for example with rollers or a brush of particular shape (cylindrical), and then
in a second step, the dispersion (A) is applied to said fibres, the application method preferably being by means of a spray, and then
in a third step, said fibres are dried naturally or dried using conventional devices used in cosmetics, and then
the shaping means is removed from said fibres, optionally followed by a rinsing step, a step of shampoo washing and then a step of drying naturally or of drying using conventional devices.

Once the dispersion (A) has been applied, before the third step, rinsing or shampoo washing may optionally be performed.

The dispersion (A) may be applied to wet or dry keratin fibres, preferably dry keratin fibres.

It is also possible via the keratin fibre treatment process of the invention to perform shaping while at the same time providing said fibres with one or more cosmetic active agents, for example dyeing by applying at least one dye and/or pigment, and/or applying at least one UV (A) and/or UV (B) screening agent, and/or applying at least one active agent to said fibres. It suffices to apply a dispersion (A) comprising at least one cosmetic agent v) as defined previously. It also appears that the shaping is persistent and, what is more, that the cosmetic active agent(s) iv) applied are also persistent notably with respect to successive shampoo washing and to light rays.

After the application of the dispersion (A), the fibres may be left to dry or may be dried, for example at a temperature of greater than or equal to 30° C. According to a particular embodiment, this temperature is greater than 40° C. According to a particular embodiment, this temperature is greater than 45° C. and less than 100° C.

Preferably, if the fibres are dried, they are dried, in addition to a supply of heat, with a flow of air obtained using a conventional device used in cosmetics, such as a hood, a hairdryer, a straightening iron, a Climazon, etc.

During drying, a mechanical action may be exerted on the locks, such as combing, brushing or running the fingers through. This operation may similarly be performed once the fibres have dried, naturally or otherwise.

When the drying step is performed with a hood or a hairdryer, the drying temperature is between 40° C. and 110° C. and preferably between 50° C. and 90° C.

According to one embodiment of the keratin fibre treatment process of the invention, the hair undergoes a treatment with a straightening iron. This treatment is then performed once said hair is dry; the temperature of the treatment with the straightening iron is between 110° C. and 220° C., preferably between 140° C. and 200° C.

The dispersion (A) may be used on wet or dry keratin fibres, and also on any type of fair or dark, natural or dyed, permanent-waved, bleached or relaxed fibres.

According to a particular embodiment of the process of the invention, the fibres are washed before applying the dispersion (A).

The application to the fibres may be performed via any standard means, in particular using a comb, a fine brush, a coarse brush or with the fingers.

According to one preferred embodiment of the invention, the step of applying the dispersion (A) is performed on dry keratin fibres.

According to another particular embodiment of the process of the invention, the step of applying the dispersion (A) is performed on humid or wet keratin fibres.

Preferably, after applying the dispersion (A), there is a waiting time of between 1 minute and 2 hours, in particular between 5 minutes and 1 hour, more particularly between 10 minutes and 30 minutes, preferably in the open air and at room temperature.

According to another particular embodiment of the invention, the process for treating keratin fibres, notably human keratin fibres such as the hair, is a process for dyeing said fibres, comprising at least one step of applying to said fibres a dispersion (A) as defined previously which comprises f) at least one dye, and/or g) at least one pigment, followed by a drying step. Once the application of the dispersion (A) of the invention to the keratin fibres has been performed, rinsing and/or shampoo washing may optionally be performed.

The dispersion (A) may be applied to wet or dry keratin fibres, which are preferably dried naturally or else dried using conventional devices used in cosmetics as defined previously.

According to a particular embodiment of the invention, the process for treating keratin materials is a process for treating the skin and/or the eyelashes or the eyebrows involving the application to the skin and/or the eyelashes or the eyebrows of the dispersion (A) as defined previously, followed by a step of drying naturally or of drying using conventional devices used in cosmetics as defined previously, preferably naturally.

According to a particular embodiment of the invention, the process for treating keratin materials is a process for making up the skin and/or the eyelashes or the eyebrows involving a step of applying the dispersion (A) comprising at least one dye f) and/or at least one pigment g), preferably at least one pigment.

The dispersion (A) according to the invention may also comprise a cosmetic additive chosen from fragrances, preserving agents, fillers, waxes, moisturizers, vitamins, ceramides, antioxidants, free-radical scavengers, polymers other than a), b), c), d) and e), thickeners, and dyestuffs such as direct dyes or pigments.

Preferentially, the first step of the process of the invention is the application of the dispersion (A) in one or more apolar solvents, notably isododecane.

### The Kit

A subject of the invention is also a kit or device with several separate compartments comprising:
in one compartment: a dispersion comprising the ingredients i) to iii) as defined previously,
the following ingredients are distributed among one or more different compartments: f) dyes, g) pigments; h) active agents for caring for keratin materials, notably the skin, and/or j) UV (A) and/or UV (B) screening agents, and iv) water, which is in the dispersion, and/or with the ingredients f) to h) and/or in another compartment.

The composition packaging assembly is, in a known manner, any packaging that is suitable for storing cosmetic compositions (notably a bottle, tube, spray bottle or aerosol can).

The invention is illustrated in greater detail in the examples that follow.

### EXAMPLES

The following dispersions (A) were prepared:

TABLE 1
Weight composition of the various illustrated
dispersions of the invention

| | Polymer | | |
|---|---|---|---|
| Examples | particles (i + ii) | Isododecane (iii) | Water (iv) |
| Example 1 | 30 | 60 | 10 |
| Example 2-1 | 30 | 50 | 20 |
| Example 2-2 | 30 | 30 | 40 |

The monomers used for the various examples for i), ii) and the polymerization initiator vi) are all collated in Tables 2 and 3 below:

TABLE 2
Monomers used in the various examples

| Monomers | CAS | Supplier |
|---|---|---|
| Stearyl methacrylate | 32360-05-7 | Sigma Aldrich |
| Methyl acrylate | 96-33-3 | Sigma Aldrich |
| Ethyl acrylate | 140-88-5 | Sigma Aldrich |
| Acrylic acid | 79-10-7 | Sigma Aldrich |

TABLE 3
Polymerization initiator used in the various examples

| Radical initiator | CAS | Supplier |
|---|---|---|
| Trigonox 21S | 3006-82-4 | AkzoNobel |

For all the dispersions, an evaluation of the cosmetic properties on a dry film was performed.

A film is prepared on a contrast card with a film spreader (speed: 50 mm/s—Cylinder: 100 μm). The film is left to dry for 24 hours at room temperature. Once dry, the film has a thickness of about 30-50 μm, FIG. 1.

An evaluation of the resistance to the attacking factors water/olive oil/sebum is performed on the dry film:

### Measurement of the Resistance to Water and to Fats

Three drops of olive oil or of sebum were placed on the dry film present on the black part of the contrast card (one for each time of observation of the resistance to the attacking factor), each drop corresponds to about 30 μL of attacking factor (use of a micropipette).

The drop is left in contact with the dry film for 20 minutes. Once the time has elapsed, the drop of olive oil, sebum or water is wiped off and observation of the deterioration of the polymer film is performed. If the film was attacked by the drop of attacking factor, the polymer film is regarded as being non-resistant to this attacking factor.

### Example 1: Production of a Dispersion with Variable Amounts of Water

Step 1: Synthesis of the dispersion of polymer particles in isododecane. In a first stage, the polymer particles are synthesized in isododecane.

The polymer particles are formed as a whole (stabilizer ii+particles i) containing 94.5% methyl acrylate and 5.5% stearyl methacrylate. The synthesis of these dispersions was performed in a 6-litre pilot reactor. The synthesis is performed in two steps:
In a first step, stearyl methacrylate is polymerized in isododecane/ethyl acetate (60/40) in the presence of a small amount of methyl acrylate and of a radical initiator (T21S). In the first step, the stearyl methacrylate/methyl acrylate mass ratio is 85/15.
In the second step, the rest of the methyl acrylate is added in the presence of isododecane/ethyl acetate (60/40) and of the radical initiator (T21S).

After stripping, the polymer is at a solids content of 50% in the isododecane.

The ratios employed to obtain the stabilizer and the core are summarized in Table 4:

TABLE 4
Specific ratios in the stabilizer ii) and core
i) for the polymer particles of Example 1

| | | | Mass percentage in |
|---|---|---|---|
| Particle | Mass percentage | | the stabilizer |
| i) + ii) | Stabilizer - Core | Monomers | and the core in g. |
| Stabilizer | 6.4 | Stearyl | 85 |
| ii) | | methacrylate | |
| | | Methyl acrylate | 15 |
| Core i) | 93.6 | Methyl acrylate | 100 |

### Amount of Reagents:

### Step 1.1:

TABLE 5

| Reagents | Mass (g) |
|---|---|
| Stearyl methacrylate | 82.5 |
| Methyl acrylate | 14.04 |
| T21S | 0.96 |
| Isododecane/EtOAc (60/40) | 360 |

Isododecane/ethyl acetate added between the two steps:

TABLE 6

| Reagent | Mass (g) |
|---|---|
| Isododecane/EtOAc (60/40) | 1200 |

### Step 1.2:

TABLE 7
Amounts of reagents used for Example 1 - Step 1

| | | Mass added to the beaker |
|---|---|---|
| Reagents | Mass (g) | for the addition (g) |
| Methyl acrylate | 1404 | 1614.6 |
| T21S | 14.04 | 16.14 |
| Isododecane/EtOAc | 1404 | 1614.6 |
| (60/40) | | |

### Experimental Protocol:

Isododecane/ethyl acetate (60/40), stearyl methacrylate, methyl acrylate and T21S are introduced as feedstock into the reactor. The medium is heated to 90° C. (nominal medium temperature) under argon and with stirring.

After heating for 2 hours, NMR indicates a stearyl methacrylate consumption of 98% (methyl acrylate consumption: 91%).

After 2 hours of reaction, isododecane/ethyl acetate (60/40) is introduced into the feedstock. The medium is heated to 90° C.

Once the medium is at 90° C., methyl acrylate, isododecane/ethyl acetate (60/40) and T21S are introduced over 2 hours by pouring. At the end of the introduction by pouring, the medium is milky.

After 7 hours of synthesis, a 99% consumption of the methyl acrylate is obtained (stearyl methacrylate consumption: 100%).

3 L of isododecane and of ethyl acetate are then stripped out (NMR indicates that there are no more monomers and that the ethyl acetate has been totally removed from the dispersion). The solids content is 50%.

### Step 2: Addition of Water at the End of the Synthesis

In a second stage, water is added at the end of the operation while keeping the polymer in the reactor. A test was performed. It is summarized in Table 8:

TABLE 8

| | Mass percentage of active material in |
|---|---|
| Example 1 | the final dispersion (A) in grams (g) |
| Polymer particle | 30 |
| Isododecane | 60 |
| Water | qs 100 |
| Appearance of | Homogeneous white formulation, |
| the dispersion | stable at 27° C. |

Evaluation of the cosmetic properties of the dispersion was subsequently performed. The measurements of the sensitivity to fatty substances (olive oil and sebum) and to water are collated in Table 9:

TABLE 9
Cosmetic properties of Example 1

| | Resistance to | Resistance to | Resistance to |
|---|---|---|---|
| Example No. | water | olive oil | sebum |
| Example 1-1 | ++ | ++ | ++ |

(−): no resistance, (+) resistance, (++), very high resistance after wiping with the same cotton fabric soaked with the same amount of sebum, olive oil or water.

The dispersion containing less volatile hydrocarbon-based oil iii) of Example 1 leads to a deposit which is resistant to water, to olive oil and to sebum.

### Example 2

### Step 1: Synthesis of the Dispersion of Polymer Particles in Isododecane

In a first stage, the polymer particles are synthesized in isododecane.

The dispersions are formed as a whole (stabilizer ii+particles i) containing 10% acrylic acid, 30% ethyl acrylate, 54.5% methyl acrylate and 5.5% stearyl methacrylate. The synthesis of these dispersions was performed in a 6-litre pilot reactor. The synthesis is performed in two steps:
In a first step, stearyl methacrylate is polymerized in isododecane/ethyl acetate (60/40) in the presence of a small amount of methyl acrylate/ethyl acrylate and of a radical initiator (T21S). In the first step, the stearyl methacrylate/methyl acrylate/ethyl acrylate mass ratio is 85/7.5/7.5.
In the second step, the rest of the methyl acrylate, the ethyl acrylate and the acrylic acid are added in the presence of isododecane/ethyl acetate (60/40) and of the radical initiator Trigonox (T21S).

After stripping, the polymer is at a solids content of 50% in the isododecane.

The ratios employed to obtain the stabilizer and the core are summarized in Table 10:

TABLE 10

| | | | Mass percentage in |
|---|---|---|---|
| | Mass percentage | | the stabilizer |
| | Stabilizer - Core | Monomer | and the core |
| Stabilizer ii) | 6.4 | Stearyl | 85 |
| | | methacrylate | |
| | | Methyl acrylate | 7.5 |
| | | Ethyl acrylate | 7.5 |
| Core i) | 93.6 | Methyl acrylate | 58 |
| | | Ethyl acrylate | 31 |
| | | Acrylic acid | 11 |

### Amount of Reagents:

### Step 1.1:

TABLE 11

| Reagents | Mass (g) |
|---|---|
| Stearyl methacrylate | 82.5 |
| Methyl acrylate | 7.02 |
| Ethyl acrylate | 7.02 |
| T21S | 5.79 |
| Isododecane/EtOAc | 360 |
| (60/40) | |

### Isododecane/Ethyl Acetate Added Between the Two Steps:

TABLE 12

| Reagent | Mass (g) |
|---|---|
| Isododecane/EtOAc | 1200 |
| (60/40) | |

### Step 1.2:

TABLE 12
Amounts of reagents used for Example 2 - Step 1

| | | Mass added to the beaker |
|---|---|---|
| Reagents | Mass (g) | for the addition (g) |
| Methyl acrylate | 810.6 | 932.2 |
| Ethyl acrylate | 442.98 | 509.43 |
| Acrylic acid | 150 | 172.5 |
| T21S | 84.21 | 96.85 |
| Isododecane/EtOAc | 1404 | 1614.6 |
| (60/40) | | |

### Experimental Protocol:

Isododecane/ethyl acetate (60/40), stearyl methacrylate, methyl acrylate, ethyl acrylate and T21S are introduced as feedstock into the reactor. The medium is heated to 90° C. (nominal medium temperature) under argon and with stirring.

After heating for 2 hours, NMR indicates a stearyl methacrylate consumption of 95%.

After 2 hours of reaction, isododecane/ethyl acetate (60/40) is introduced into the feedstock. The medium is heated to 90° C.

Once the medium is at 90° C., methyl acrylate/ethyl acrylate/acrylic acid, isododecane/ethyl acetate (60/40) and the T21S are introduced over 1 hour by pouring. At the end of the introduction by pouring, the medium is milky.

After 7 hours of synthesis, traces of the starting monomers remain.

2.8 L of isododecane and of ethyl acetate is then stripped out (NMR indicates that there are no more monomers and that the ethyl acetate has been totally removed from the dispersion). The solids content is 50%.

### Step 2: Addition of Water at the End of the Synthesis

In a second stage, water is added at the end of the operation while keeping the polymer in the reactor. Two tests were performed with introduction of 20% and 40% of water. They are summarized in Table 13:

TABLE 13
dispersions containing acrylic acid - Example 2

Mass percentage of active
material in the final dispersion

| Ingredients | Example 2-1 | Example 2-2 |
|---|---|---|
| Polymer particle | 30 | 30 |
| Isododecane | 50 | 30 |
| Water | 20 | 40 |
| Appearance of the | Homogeneous white | Homogeneous white |
| formulations | formulation | formulation |

Two stable dispersions containing 20% and 40% of water were obtained.

Evaluation of the cosmetic properties of the new dispersions was subsequently performed. The measurements of the sensitivity to fatty substances (olive oil and sebum) and to water are collated in Table 14:

TABLE 14
Cosmetic properties of Examples 2-1 and 2-2

| | Resistance to | Resistance to | Resistance to |
|---|---|---|---|
| Example No. | water | olive oil | sebum |
| Example 2-1 | ++ | ++ | ++ |
| Example 2-2 | ++ | ++ | ++ |

(−): no resistance, (+) resistance, (++), very high resistance after wiping with the same cotton fabric soaked with the same amount of sebum, olive oil or water.

The dispersions containing a smaller amount of volatile hydrocarbon-based oil such as isododecane of Example 2, Ex. 2-1 (50%) vs. Ex. 2-2 (20%) lead to deposits which remain very resistant to water, to olive oil and to sebum.

1. A dispersion (A) which comprises:
i) one or more particles including one or more polymers chosen from:
a) ethylenic homopolymers of (C₁-C₄)alkyl (C₁-C₄)(alkyl)acrylate;
b) ethylenic copolymers of b1) (C₁-C₄)alkyl (C₁-C₄)(alkyl)acrylate, and of b2) ethylenic monomers comprising one or more groups from among carboxyl, anhydride, phosphoric acid, sulfonic acid and/or aryl such as benzyl;
c) ethylenic copolymers of (C₁-C₄)alkyl (C₁-C₄)(alkyl)acrylate alkyl; and
ii) one or more polymeric stabilizers chosen from:
d) ethylenic homopolymers of (C₉-C₂₂)alkyl (C₁-C₆)(alkyl)acrylate; and
e) ethylenic copolymers of e1) (C₉-C₂₂)alkyl (C₁-C₆)(alkyl)acrylate, and of e2) (C₁-C₄)alkyl (C₁-C₄)(alkyl)acrylate;
iii) one or more hydrocarbon-based liquid fatty substances;
iv) water; and
v) optionally one or more cosmetic active agents chosen from f) dyes, g) pigments; h) active agents for caring for keratin materials, and j) UV (A) and/or (B) screening agents, and m) mixtures thereof;
it being understood that the amount of water in the dispersion (A) is greater than or equal to 2% by weight and less than 50% by weight relative to the total weight of the dispersion.

2. The dispersion (A) according to claim 1, in which the particle(s) i) consist of an ethylenic polymer core derived from homopolymers a) or from copolymers b) or c), and ii) of one or more polymeric surface stabilizers derived from the homopolymer d) and the copolymers e).

3. The dispersion (A) according to claim 1, in which the polymer(s) constituting the particles i) are chosen from ethylenic acrylate homopolymers c) resulting from the polymerization of an identical monomer of formula (I):
H₂C═C(R)—C(O)—O—R′ (I)
in which formula (I):
R represents a hydrogen atom or a (C₁-C₄)alkyl group, and
R′ represents a (C₁-C₄)alkyl group.

4. The dispersion (A) according to claim 1, in which the polymer(s) constituting the particles i) are chosen from ethylenic copolymers of b1) (C₁-C₄)alkyl (C₁-C₄)(alkyl)acrylate and of b2) ethylenic monomers comprising one or more groups from among carboxyl, anhydride, phosphoric acid, sulfonic acid and/or aryl):
(1) R₁(R₂)C═C(R₃)-Acid with R₁, R₂ and R₃ representing a hydrogen atom or a CO₂H, H₂PO₄ or SO₃H group, and Acid representing a carboxyl, phosphoric acid or sulfonic acid;
(2) H₂C═C(R)—C(O)—N(R′)-Alk-Acid with R and R′, which may be identical or different, representing a hydrogen atom or a (C₁-C₄)alkyl group; Alk represents a (C₁-C₆)alkylene group optionally substituted with at least one group chosen from Acid as defined previously and hydroxyl; and Acid is as defined previously;
(3) Ar—(Rₐ)C═C(R_{b})—R_{c} with Rₐ, R_{b} and R_{c}, which may be identical or different, representing a hydrogen atom or a (C₁-C₄)alkyl group, and Ar representing an aryl group, optionally substituted with at least one acid group CO₂H, H₂PO₄ or SO₃H;
(4) maleic anhydride of formulae (4a) and (4b): 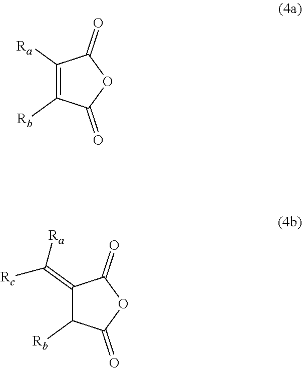
in which formulae (4a) and (4b) Rₐ, R_{b} and R_{c}, which may be identical or different, represent a hydrogen atom or a (C₁-C₄)alkyl group.

5. The dispersion (A) according to claim 1, in which the polymer(s) constituting the particles i) are chosen from ethylenic acrylate copolymers b) derived from the polymerization:
of at least two different monomers of formula (I)
H₂C═C(R)—C(O)—O—R′ (I), wherein R represents a hydrogen atom or a (C₁-C₄)alkyl group, and
R′ presents a (C₁-C₄)alkyl group; and
optionally of a monomer of formula (II) H₂C═C(R)—C(O)—O—H with R representing a hydrogen atom or a (C₁-C₄)alkyl group.

6. The dispersion (A) according to claim 1, in which the dispersion (A) includes from 60% to 98% by weight of monomers a) to c) relative to the total weight of polymers i) contained in said dispersion.

7. The dispersion (A) according to claim 1, in which the stabilizer(s) ii) are chosen from d) ethylenic homopolymers of d) (C₉-C₂₂)alkyl (C₁-C₆)(alkyl)acrylate.

8. The dispersion (A) according to claim 1, in which the stabilizer(s) ii) are chosen from e) ethylenic copolymers of (C₉-C₂₂)alkyl (C₁-C₆)(alkyl)acrylate and of (C₁-C₄)alkyl (C₁-C₄)(alkyl)acrylate.

9. The dispersion (A) according to claim 1, in which the stabilizer(s) ii) are chosen from ethylenic copolymers e) derived from the polymerization e1) of a monomer of H₂C═C(R)—C(O)—O—R″ (IV) and e2) two different monomers of H₂C═C(R)—C(O)—O—R′ (III); in which formulae (III) and (IV):
R, which may be identical or different, represents a hydrogen atom or a (C₁-C₄)alkyl group;
R′, which may be identical or different, represents a (C₁-C₄)alkyl group; and
R″ represents a (C₉-C₂₂)alkyl.

10. The dispersion (A) according to claim 1, in which the dispersion (A) includes from 2% to 40% by weight of (C₉-C₂₂)alkyl (C₁-C₆)(alkyl)acrylate monomers included in d) or e) with the hydrocarbon-based liquid fatty substance(s) iii), relative to the total weight of polymers contained in said dispersion.

11. The dispersion (A) according to claim 1, in which the ii) stabilizer(s)+i) polymer particle(s) present in the dispersion (A) together comprises from 2% to 40% by weight by weight of the stabilizer(s) relative to the total weight of monomers used (stabilizers ii)+polymer particles i)).

12. The dispersion (A) according to claim 1, in which the hydrocarbon-based liquid fatty substance(s) iii) are chosen from hydrocarbons, oils of animal origin, oils of plant origin, glycerides or fluoro oils of synthetic origin, fatty alcohols, esters of fatty acid and/or of fatty alcohol, non-silicone waxes and silicones.

13. The dispersion (A) according to claim 1, in which the hydrocarbon-based liquid fatty substance(s) iii) are chosen from:
apolar oils, i.e. oils formed solely from carbon and hydrogen atoms.

14. The dispersion (A) according to claim 1, in which the hydrocarbon-based liquid fatty substance(s) iii) are present in the dispersion in a content of between 15% by weight and 80% by weight.

15. The dispersion (A) according to claim 1, in which the amount of water iv) in the dispersion is between 5% and 49% by weight relative to the total weight of the dispersion.

16. The dispersion (A) according to claim 1, in which said dispersion does not comprise more than 3% by weight of surfactants relative to the total weight of the dispersion.

17. The dispersion (A) according to claim 1, in which the cosmetic active agent(s) v) are chosen from:
organic and mineral or inorganic pigments,
pigments with special effects or mixtures thereof;
said pigments may be dispersed in the product by means of a dispersant and the pigments may be surface-treated with an organic agent.

18. A process for treating keratin materials comprising applying to said materials at least one dispersion (A) as defined in claim 1.

19. A kit or device containing several separate compartments, comprising:
in one compartment: the dispersion (A) comprising the ingredients i) to iii), and
the following ingredients distributed among one or more different compartments: f) dyes, g) pigments; h) active agents for caring for keratin materials and/or j) UV (A) and/or (B) screening agents, wherein ingredients i) to iii), f) dyes, g) pigments; h) active agents for caring for keratin materials and/or j) UV (A) and/or (B) screening agents are as defined in claim 1.

20. A process for dyeing keratin fibres and/or for shaping keratin fibres or for making up human skin which comprises applying a dispersion (A) as defined in claim 1 to the keratin fibres or the human skin.
